(19) United States
(12) Patent Application Publication
(10) Pub. No.: US 2017/0298073 A1
(43) Pub. Date: Oct. 19, 2017

(54) SALTS, CO-CRYSTALS, AMORPHOUS FORMS, AND CRYSTALLINE FORMS OF A CO-ACTIVATOR-ASSOCIATED ARGININE METHYLTRANSFERASE 1 (CARM1) INHIBITOR

(71) Applicant: Epizyme, Inc, Cambridge, MA (US)

(72) Inventors: Edward James Olhava, Newton, MA (US); Oscar Miguel Moradei, Burlington, MA (US); Gideon Shapiro, Gainesville, FL (US); Richard Chesworth, Concord, MA (US); Lei Jin, Wellesley, MA (US); Robert E. Babine, Carlsbad, CA (US)

(73) Assignee: Epizyme, Inc, Cambridge, MA (US)

(21) Appl. No.: 15/511,520

(22) PCT Filed: Sep. 17, 2015
(86) PCT No.: PCT/US15/50647

§ 371 Date: Mar. 15, 2017

Related U.S. Application Data
(60) Provisional application No. 62/051,878, filed on Sep. 17, 2014.

(51) Int. Cl. C07D 487/10; C07C 65/05

(52) U.S. Cl. CPC C07D 487/10; C07C 65/05; C07B 2200/13

(57) ABSTRACT

Provided herein are solid forms (e.g., salts, co-crystals, amorphous forms, and crystalline forms) of methyl (R)-2-(2-(2-chloro-5-(2-hydroxy-3-(methylamino)propoxy)phenyl)-6-(3,5-dimethylisoxazol-4-yl)-5-methylpyrimidin-4-yl)-2,7-diazaspiro[3.5]nonane-7-carboxylate (compound 109-3). Also provided are pharmaceutical compositions, kits, methods, and uses that include or involve the solid forms for inhibiting the activity of co-activator-associated arginine methyltransferase 1 (CARM1) and for treating CARM1-mediated disorders (e.g., proliferative disorders and metabolic disorders).

### RELATED APPLICATIONS

The present application claims priority under 35 U.S.C. §119(e) to U.S. provisional patent application, U.S. Ser. No. 62/051,878, filed Sep. 17, 2014, the entire contents of which is incorporated herein by reference.

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., CARM1 (co-activator-associated arginine methyltransferase 1; PRMT4)), many of which are associated with specific genetic alterations that can cause human disease.

Disease-associated chromatin-modifying enzymes play a role in diseases such as proliferative disorders, autoimmune disorders, muscular disorders, and neurological disorders. Thus, there is a need for the development of small molecules that are capable of inhibiting the activity of CARM1.

### SUMMARY OF THE INVENTION

In one aspect, described herein are Embodiments B1-B8:

Embodiment B1

A crystalline form G-C (Form G-C) of compound 109-3 of the formula:

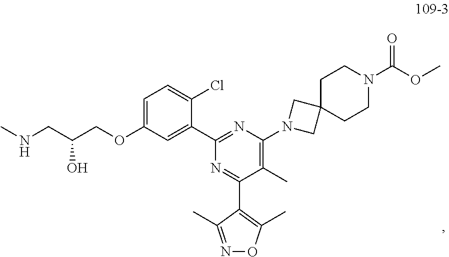

wherein the crystalline form G-C comprises gentisic acid.

Embodiment B2

The crystalline form G-C of Embodiment B1, wherein the crystalline form G-C is characterized by an XRPD pattern comprising three or more characteristic peaks, expressed in degrees 2-theta (±0.2), independently selected from the group consisting of 17.86, 19.44, 19.24, 18.65, 20.49, 23.88, 11.43, 15.83, and 23.45.

Embodiment B3

The crystalline form G-C of Embodiment B1, wherein the crystalline form G-C is characterized by an XRPD pattern comprising four or more characteristic peaks, expressed in degrees 2-theta (±0.2), independently selected from the group consisting of 17.86, 19.44, 19.24, 18.65, 20.49, 23.88, 11.43, 15.83, and 23.45.

Embodiment B4

The crystalline form G-C of Embodiment B1, wherein the crystalline form G-C is characterized by an XRPD pattern comprising five or more characteristic peaks, expressed in degrees 2-theta (±0.2), independently selected from the group consisting of 17.86, 19.44, 19.24, 18.65, 20.49, 23.88, 11.43, 15.83, and 23.45.

Embodiment B5

The crystalline form G-C of Embodiment B1, wherein the crystalline form G-C is characterized by an XRPD pattern comprising six or more characteristic peaks, expressed in degrees 2-theta (±0.2), independently selected from the group consisting of 17.86, 19.44, 19.24, 18.65, 20.49, 23.88, 11.43, 15.83, and 23.45.

Embodiment B6

The crystalline form G-C of Embodiment B1, wherein the crystalline form G-C is characterized by an XRPD pattern comprising seven or more characteristic peaks, expressed in degrees 2-theta (±0.2), independently selected from the group consisting of 17.86, 19.44, 19.24, 18.65, 20.49, 23.88, 11.43, 15.83, and 23.45.

Embodiment B7

The crystalline form G-C of Embodiment B1, wherein the crystalline form G-C is characterized by an XRPD pattern comprising eight or more characteristic peaks, expressed in degrees 2-theta (±0.2), independently selected from the group consisting of 17.86, 19.44, 19.24, 18.65, 20.49, 23.88, 11.43, 15.83, and 23.45.

Embodiment B8

The crystalline form G-C of any one of embodiments B1-B7, wherein the crystalline form G-C is characterized by a differential scanning calorimetry (DSC) thermogram comprising an endotherm comprising an onset temperature (Tₘ) of 154.4±2° C.

In another aspect, described herein are Embodiments C1-C8:

Embodiment C1

A crystalline form G-A (Form G-A) of compound 109-3, wherein the crystalline form G-A comprises gentisic acid.

Embodiment C2

The crystalline form G-A of Embodiment C1, wherein the crystalline form G-A is characterized by an XRPD pattern comprising three or more characteristic peaks, expressed in degrees 2-theta (±0.2), independently selected from the group consisting of 20.51, 13.37, 9.13, 19.64, 13.04, 19.86, 18.30, 9.49, and 18.68.

Embodiment C3

The crystalline form G-A of Embodiment C1, wherein the crystalline form G-A is characterized by an XRPD pattern comprising four or more characteristic peaks, expressed in degrees 2-theta (±0.2), independently selected from the group consisting of 20.51, 13.37, 9.13, 19.64, 13.04, 19.86, 18.30, 9.49, and 18.68.

Embodiment C4

The crystalline form G-A of Embodiment C1, wherein the crystalline form G-A is characterized by an XRPD pattern comprising five or more characteristic peaks, expressed in degrees 2-theta (±0.2), independently selected from the group consisting of 20.51, 13.37, 9.13, 19.64, 13.04, 19.86, 18.30, 9.49, and 18.68.

Embodiment C5

The crystalline form G-A of Embodiment C1, wherein the crystalline form G-A is characterized by an XRPD pattern comprising six or more characteristic peaks, expressed in degrees 2-theta (±0.2), independently selected from the group consisting of 20.51, 13.37, 9.13, 19.64, 13.04, 19.86, 18.30, 9.49, and 18.68.

Embodiment C6

The crystalline form G-A of Embodiment C1, wherein the crystalline form G-A is characterized by an XRPD pattern comprising seven or more characteristic peaks, expressed in degrees 2-theta (±0.2), independently selected from the group consisting of 20.51, 13.37, 9.13, 19.64, 13.04, 19.86, 18.30, 9.49, and 18.68.

Embodiment C7

The crystalline form G-A of Embodiment C1, wherein the crystalline form G-A is characterized by an XRPD pattern comprising eight or more characteristic peaks, expressed in degrees 2-theta (±0.2), independently selected from the group consisting of 20.51, 13.37, 9.13, 19.64, 13.04, 19.86, 18.30, 9.49, and 18.68.

Embodiment C8

The crystalline form G-A of any one of embodiments C1-C7, wherein the crystalline form G-A is characterized by a differential scanning calorimetry (DSC) thermogram comprising an endotherm comprising an onset temperature (Tₘ) of 178.3±2° C.

In another aspect, described herein are Embodiments D1-D8:

Embodiment D1

A crystalline form G-B (Form G-B) of compound 109-3, wherein the crystalline form G-B comprises gentisic acid.

Embodiment D2. The crystalline form G-B of Embodiment D1, wherein the crystalline form G-B is characterized by an XRPD pattern comprising three or more characteristic peaks, expressed in degrees 2-theta (±0.2), independently selected from the group consisting of 17.53, 15.87, 10.57, 20.92, 22.22, 18.67, 21.21, 20.34, and 27.06.

Embodiment D3. The crystalline form G-B of Embodiment D1, wherein the crystalline form G-B is characterized by an XRPD pattern comprising four or more characteristic peaks, expressed in degrees 2-theta (±0.2), independently selected from the group consisting of 17.53, 15.87, 10.57, 20.92, 22.22, 18.67, 21.21, 20.34, and 27.06.

Embodiment D4. The crystalline form G-B of Embodiment D1, wherein the crystalline form G-B is characterized by an XRPD pattern comprising five or more characteristic peaks, expressed in degrees 2-theta (±0.2), independently selected from the group consisting of 17.53, 15.87, 10.57, 20.92, 22.22, 18.67, 21.21, 20.34, and 27.06.

Embodiment D5. The crystalline form G-B of Embodiment D1, wherein the crystalline form G-B is characterized by an XRPD pattern comprising six or more characteristic peaks, expressed in degrees 2-theta (±0.2), independently selected from the group consisting of 17.53, 15.87, 10.57, 20.92, 22.22, 18.67, 21.21, 20.34, and 27.06.

Embodiment D6. The crystalline form G-B of Embodiment D1, wherein the crystalline form G-B is characterized by an XRPD pattern comprising seven or more characteristic peaks, expressed in degrees 2-theta (±0.2), independently selected from the group consisting of 17.53, 15.87, 10.57, 20.92, 22.22, 18.67, 21.21, 20.34, and 27.06.

Embodiment D7. The crystalline form G-B of Embodiment D1, wherein the crystalline form G-B is characterized by an XRPD pattern comprising eight or more characteristic peaks, expressed in degrees 2-theta (±0.2), independently selected from the group consisting of 17.53, 15.87, 10.57, 20.92, 22.22, 18.67, 21.21, 20.34, and 27.06.

Embodiment D8. The crystalline form G-B of any one of embodiments D1-D7, wherein the crystalline form G-B is characterized by a differential scanning calorimetry (DSC) thermogram comprising an endotherm comprising an onset temperature (Tₘ) of 173.4±2° C.

Other aspects of the disclosure are provided herein.

### BRIEF DESCRIPTION OF THE DRAWINGS

FIG. 1A depicts an exemplary X-Ray Powder Diffraction (XRPD) pattern of Form A.

FIG. 1B depicts an exemplary differential scanning calorimetry (DSC) thermogram of Form A (bottom-left curve) and a thermogravimetric analysis (TGA) thermogram of Form A (top-right curve).

FIG. 1C depicts an exemplary proton nuclear magnetic resonance (¹H-NMR) spectrum of Form A in CDCl₃.

FIG. 2A depicts exemplary XRPD patterns of Form G-A (Samples 807304-06-D22, 807304-06-C22, 807304-06-B22, and 807304-06-A22).

FIG. 2B depicts an exemplary DSC thermogram of Form G-A (bottom-left curve) and a TGA thermogram of Form G-A (top-right curve).

FIG. 2C depicts an exemplary overlay of a DSC thermogram of Form G-A (top curve) and a DSC thermogram of a mixture of Form A and gentisic acid (bottom curve). The differences between the DSC thermograms indicate that Form G-A is different from Form A.

FIG. 2D depicts an exemplary overlay of XRPD patterns of Form G-A before (bottom curve) and after (top curve) heating to 100° C. and cooling to room temperature (heating-cooling). After the heating-cooling, Form G-A was converted to Form G-B.

FIG. 2E depicts another exemplary overlay of XRPD patterns of Form G-A before (bottom curve) and after (top curve) heating to 100° C. and cooling to room temperature (heating-cooling). After the heating-cooling, Form G-A was converted to Form G-B.

FIG. 2F depicts an exemplary ¹H-NMR spectrum of Form G-A in DMSO-d₆. The molar ratio of gentisic acid to compound 109-3 in Form G-A was calculated to be about 1:1 according to the ¹H NMR spectrum.

FIG. 3A depicts an exemplary XRPD pattern of Form G-B.

FIG. 3B depicts an exemplary TGA thermogram of Form G-B.

FIG. 3C depicts an exemplary DSC thermogram of Form G-B (bottom-left curve) and another exemplary TGA thermogram of Form G-B (top-right curve).

FIG. 4A depicts an exemplary XRPD pattern of Form G-C.

FIG. 4B depicts an exemplary DSC thermogram of Form G-C (bottom-left curve) and a TGA thermogram of Form G-C (top-right curve).

FIG. 4C depicts an exemplary overlay of XRPD patterns of Form G-A, Form G-B, and Form G-C.

### DETAILED DESCRIPTION OF CERTAIN EMBODIMENTS

Co-activator-associated arginine methyltransferase 1 (CARM1) is an attractive target for modulation given its role in the regulation of diverse biological processes. Various salts, co-crystals, amorphous forms, and crystalline forms of methyl (R)-2-(2-(2-chloro-5-(2-hydroxy-3-(methylamino)propoxy)phenyl)-6-(3,5-dimethylisoxazol-4-yl)-5-methylpyrimidin-4-yl)-2,7-diazaspiro[3.5]nonane-7-carboxylate (compound 109-3) have now been discovered as described herein and have been found to inhibit CARM1.

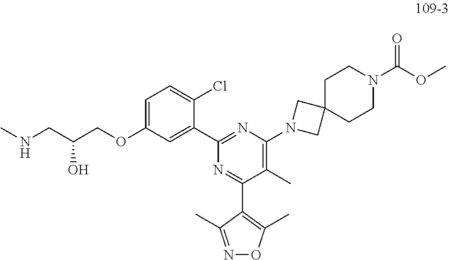

Compound 109-3 is described in International PCT Application, PCT/US2014/028463, filed Mar. 14, 2014, which is incorporated herein by reference. Salts, co-crystals, amorphous forms, and crystalline forms of compound 109-3, and pharmaceutical compositions thereof, are useful in treating and/or preventing CARM1-mediated disorders (e.g., proliferative disorders and metabolic disorders).

In one aspect, the present disclosure provides solid forms (e.g., salts, co-crystals, amorphous forms, and crystalline forms) of compound 109-3. In certain embodiments, provided herein are salts (e.g., gentisates, such as monogentisates) of compound 109-3.

The salts described herein may be in amorphous or crystalline form. The salts may be solvates (e.g., hydrates, methanolates, acetonitrile solvates, acetone solvates, and THF solvates) or may not contain any solvent. In certain embodiments, the salts are substantially anhydrous.

In certain embodiments, provided herein are co-crystals of compound 109-3. The co-crystals described herein may be in amorphous or crystalline form. In certain embodiments, the co-crystals comprise compound 109-3 and gentisic acid. The co-crystals may be solvates (e.g., hydrates, methanolates, acetonitrile solvates, acetone solvates, and THF solvates) or may not contain any solvent. In certain embodiments, the co-crystal is substantially anhydrous.

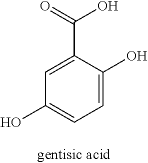 [SMILES: O=C(O)c1cc(O)ccc1O]

In certain embodiments, provided herein are amorphous forms (e.g., Form A) of compound 109-3. The amorphous forms may be solvates (e.g., hydrates) or may not contain any solvent. In certain embodiments, the amorphous form is substantially anhydrous.

In certain embodiments, provided herein are crystalline forms of compound 109-3. Crystalline forms of salts and co-crystals of compound 109-3 are also provided. In certain embodiments, a crystalline form described herein comprises compound 109-3 and gentisic acid. In certain embodiments, a crystalline form described herein is Form G-A or Form G-B. The crystalline forms may be solvates (e.g., hydrates, methanolates, acetonitrile solvates, acetone solvates, and THF solvates) or may not contain any solvent. In certain embodiments, the crystalline form is substantially anhydrous.

The salts, co-crystals, amorphous forms, and crystalline forms described herein may inhibit the activity of CARM1.

In another aspect, described herein are compositions that comprise a salt, co-crystal, amorphous form, or crystalline form described herein. In another aspect, described herein are pharmaceutical compositions that comprise a salt, co-crystal, amorphous form, or crystalline form described herein, and optionally a pharmaceutically acceptable excipient. In certain embodiments, the salt, co-crystal, amorphous form, or crystalline form is provided in an effective amount in a pharmaceutical composition described herein.

In another aspect, provided herein are methods of inhibiting CARM1, the methods comprise contacting CARM1 with an effective amount of a salt, co-crystal, amorphous form, crystalline form, or pharmaceutical composition described herein. The CARM1 may be purified or crude, and may be present in a cell, tissue, or subject. Thus, such methods encompass inhibition of CARM1 activity in vitro and/or in vivo. In certain embodiments, the CARM1 is a wild-type CARM1. In certain embodiments, the CARM1 is overexpressed. In certain embodiments, the CARM1 is a mutant. In certain embodiments, the CARM1 is in a cell. In certain embodiments, the CARM1 is in a tissue. In certain embodiments, the CARM1 is in a biological sample. In certain embodiments, the CARM1 is in an animal, e.g., a human. In some embodiments, the CARM1 is expressed at normal levels in a subject, but the subject would benefit from CARM1 inhibition (e.g., because the subject has one or more mutations in an CARM1 substrate that causes an increase in methylation of a substrate with normal levels of CARM1). In some embodiments, the CARM1 is in a subject known or identified as having abnormal or aberrant CARM1 activity (e.g., overexpression). In some embodiments, the CARM1 is in a subject known or identified as having aberrant CARM1 activity. In some embodiments, a salt, co-crystal, amorphous form, or crystalline form described herein is selective for CARM1 over other methyltransferases. In certain embodiments, a salt, co-crystal, amorphous form, crystalline form, or pharmaceutical composition described herein is at least 10-fold selective, at least 20-fold selective, at least 30-fold selective, at least 40-fold selective, at least 50-fold selective, at least 60-fold selective, at least 70-fold selective, at least 80-fold selective, at least 90-fold selective, at least 100-fold, at least 300-fold, at least 1,000-fold, at least 3,000-fold, or at least 10,000-fold selective relative to one or more other methyltransferases.

In another aspect, methods of modulating gene expression or activity in a cell are provided, the methods comprise contacting a cell with an effective amount of a salt, co-crystal, amorphous form, crystalline form, or pharmaceutical composition described herein.

In certain embodiments, the cell is in vitro (e.g., cultured in vitro). In certain embodiments, the cell is cultured in vivo. In certain embodiments, cell is in an animal, e.g., a human.

In another aspect, methods of modulating transcription in a cell are provided, the methods comprise contacting a cell with an effective amount of a salt, co-crystal, amorphous form, crystalline form, or pharmaceutical composition described herein.

In another aspect, methods of treating a CARM1-mediated disorder are provided, the methods comprise administering to a subject suffering from a CARM1-mediated disorder an effective amount of a salt, co-crystal, amorphous form, crystalline form, or pharmaceutical composition described herein. In certain embodiments, the CARM1-mediated disorder is a proliferative disorder (e.g., cancer, such as breast cancer or prostate cancer). In certain embodiments, the CARM1-mediated disorder is a metabolic disorder.

The salts, co-crystals, amorphous forms, crystalline forms, and pharmaceutical compositions described herein are also useful for the study of arginine methyltransferases (e.g., CARM1) in biological and pathological phenomena, the study of intracellular signal transduction pathways mediated by arginine methyltransferases (e.g., CARM1), and the comparative evaluation of new arginine methyltransferase (e.g., CARM1) inhibitors. The salts, co-crystals, amorphous forms, and crystalline forms described herein are also useful in the study of CARM1 in biological and pathological phenomena, the study of intracellular signal transduction pathways mediated by CARM1, and the comparative evaluation of new CARM1 inhibitors.

The total number of significant decimal digits in a number or percentage does not affect the precision and accuracy of the number or percentage. For example, the numbers “100” and “100.0” are used interchangeably.

When a characteristic peak of an X-ray powder diffraction pattern is expressed in “degrees 2-theta (±0.2)” at Z, where Z is a number, the characteristic peak is at between Z+0.2 and Z−0.2 degrees 2-theta, inclusive.

The term “about X,” where X is a number or percentage, refers to a number or percentage that is between 99.5% and 100.5%, between 99% and 101%, between 98% and 102%, between 97% and 103%, between 96% and 104%, between 95% and 105%, between 92% and 108%, or between 90% and 110%, inclusive, of X. For example, the term “about 100” or “about 100.0” refers to between 99.5 and 100.5, between 99 and 101, between 98 and 102, between 97 and 103, between 96 and 104, between 95 and 105, between 92 and 108, or between 90 and 110, inclusive.

The term “substantially Y,” where Y is a characteristic (e.g., anhydrous), refers to a characteristic that is at least 99.5%, at least 99%, at least 98%, at least 97%, at least 96%, at least 95%, at least 92%, or at least 90% the same as Y, unless expressly provided otherwise.

The term “room temperature” or “RT” refers to about 25° C. or 25±3° C. In certain embodiments, room temperature is about 25° C. In certain embodiments, room temperature is 25±3° C. (e.g., between 22 and 28° C., inclusive).

The term “salt” refers to ionic compounds that result from the neutralization reaction of an acid and a base (e.g., compound 109-3). A salt is composed of one or more cations (positively charged ions) and one or more anions (negative ions) so that the salt is electrically neutral (without a net charge). Salts of the compounds include those derived from inorganic and organic acids and bases. Examples of acid addition salts are salts of an amino group formed with inorganic acids, or organic acids, such as gentisic acid, or by using other methods known in the art such as ion exchange. Inorganic acids include hydrogen chloride, hydrochloric acid, hydrobromic acid, phosphoric acid, sulfuric acid, and perchloric acid. Additional organic acids include acetic acid, oxalic acid, maleic acid, L-tartaric acid, R-tartaric acid, citric acid, succinic acid, fumaric acid, malic acid, and malonic acid. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate. In certain embodiments, the salt is a gentisate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. In certain embodiments, a pharmaceutically acceptable salt of a compound (e.g., compound 109-3) is a salt described herein, e.g., a gentisate.

The term “solvate” refers to a form of a compound (e.g., compound 109-3), or a salt or co-crystal thereof, that is associated with a solvent, usually by a solvolysis reaction. This association may include hydrogen bonding. Conventional solvents include water, methanol, isopropanol, THF, and acetone. In certain embodiments, solvates are formed using Class 3 solvent(s). Categories of solvents are defined in, for example, the International Conference on Harmonization of Technical Requirements for Registration of Pharmaceuticals for Human Use (ICH), “Impurities: Guidelines for Residual Solvents, Q3C (R3), (November 2005). A compound (e.g., compound 109-3), or a salt or co-crystal thereof, may be prepared, e.g., in an amorphous or crystalline form, and may be solvated. Suitable solvates include pharmaceutically acceptable solvates and further include both stoichiometric solvates and non-stoichiometric solvates. In certain instances, the solvate are capable of isolation, for example, when one or more solvent molecules are incorporated in the crystal lattice of a crystalline solid. Solvates includes both solution-phase and isolable solvates. In certain embodiments, the solvate is a hydrate. In certain embodiments, the solvate is a methanolate (methanol solvate) or isopropanolate (isopropanol solvate). In certain embodiments, the solvate is an acetone solvate or THF solvate.

The term “stoichiometric solvate” refers to a solvate, which comprises a compound (e.g., compound 109-3), or a salt thereof, and a solvent, wherein the solvent molecules are an integral part of the crystal lattice, in which they interact strongly with the compound, or a salt or co-crystal thereof, and each other. The removal of the solvent molecules will cause instability of the crystal network, which subsequently collapses into an amorphous phase or recrystallizes as a new crystalline form with reduced solvent content.

The term “non-stoichiometric solvate” refers to a solvate, which comprises a compound (e.g., compound 109-3), or a salt or co-crystal thereof, and a solvent, wherein the solvent content may vary without major changes in the crystal structure. The amount of solvent in the crystal lattice only depends on the partial pressure of solvent in the surrounding atmosphere. In the fully solvated state, non-stoichiometric solvates may, but not necessarily have to, show an integer molar ratio of solvent to the compound, or salt or co-crystal thereof. During drying of a non-stoichiometric solvate, a portion of the solvent may be removed without significantly disturbing the crystal network, and the resulting solvate can subsequently be resolvated to give the initial crystalline form. Unlike stoichiometric solvates, the desolvation and resolvation of non-stoichiometric solvates is not accompanied by a phase transition, and all solvation states represent the same crystal form.

The term “hydrate” refers to a compound (e.g., compound 109-3), or a salt or co-crystal thereof, that is associated with water. Typically, the number of the water molecules contained in a hydrate of a compound, or a salt or co-crystal thereof, is in a definite ratio to the number of molecules of the compound, or a salt or co-crystal thereof, in the hydrate. Therefore, a hydrate of a compound, or a salt or co-crystal thereof, may be represented, for example, by the general formula R·x H₂O, wherein R is the compound, or salt or co-crystal thereof, and x is a number greater than 0. A given compound, or a salt or co-crystal thereof, may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “amorphous” or “amorphous form” refers to a form of a solid (“solid form”), the form lacking long-range order. In certain embodiments, an amorphous form of a solid is a solid form that is substantially not crystalline. In certain embodiments, the X-ray powder diffraction (XRPD) pattern of an amorphous form includes a wide scattering band with a peak at 2θ of, e.g., between 20 and 70° , inclusive, using CuKα radiation. In certain embodiments, the XRPD pattern of an amorphous form further includes one or more peaks attributed to crystalline structures. In certain embodiments, the maximum intensity of any one of the one or more peaks attributed to crystalline structures observed at a 2θ of between 20 and 70°, inclusive, is not more than 300-fold, not more than 100-fold, not more than 30-fold, not more than 10-fold, or not more than 3-fold of the maximum intensity of the wide scattering band. In certain embodiments, the XRPD pattern of an amorphous form includes no peaks attributed to crystalline structures.

The term “co-crystal” refers to a crystalline structure comprising at least two different components (e.g., compound 109-3 and an acid), wherein each of the components is independently an atom, ion, or molecule. In certain embodiments, none of the components is a solvent. In certain embodiments, at least one of the components is a solvent. A co-crystal comprising compound 109-3 and an acid is different from a salt formed from compound 109-3 and the acid. In a salt, compound 109-3 is complexed with the acid in a way that proton transfer (e.g., a complete proton transfer) from the acid to compound 109-3 easily occurs at room temperature. In a co-crystal, however, compound 109-3 is complexed with the acid in a way that proton transfer from the acid to compound 109-3 does not easily occur at room temperature. In certain embodiments, of a co-crystal, there is no proton transfer from the acid to compound 109-3. In certain embodiments of a co-crystal, there is partial proton transfer from the acid to compound 109-3. Co-crystals may be useful to improve the properties (e.g., solubility, stability, and ease of formulation) of compound 109-3.

The term “impurity” refers to extraneous matter included in a desired substance (e.g., a compound (e.g., compound 109-3), or a salt, solvate, hydrate, co-crystal, amorphous form, or crystalline form thereof). Extraneous matter includes one or more substances that are different from the desired substance. In certain embodiments, the extraneous matter is undesired extraneous matter. For example, when the desired substance is an anhydrous compound, a solvent (e.g., water) included in or with the anhydrous compound is an impurity. When the desired substance is a crystalline compound, an amorphous form of the compound included in or with the crystalline compound is an impurity. When the desired substance is a certain solid form of a compound, a different solid form of the compound included in or with the solid form of the compound is an impurity. When the desired substance is a salt of a compound, a different salt of the compound included in or with the salt of the compound is an impurity. The term “substantially free of impurities” means that a desired substance does not contain a significant amount of extraneous matter (e.g., undesired extraneous matter). What amount of the extraneous matter constitutes a significant amount depends on the subject matter and is understood in the art. In certain embodiments, at least 0.5%, at least 1%, at least 2%, at least 3%, at least 4%, at least 5%, or at least 10% by weight of extraneous matter in the desired substance is a significant amount of extraneous matter. The amount of impurities may be determined using high-performance liquid chromatography (HPLC) with, e.g., an ultraviolet (UV) detector at, e.g., about 214 or about 220 nm. Under suitable conditions, a desired substance and each impurity are separated after HPLC, and the areas of the peaks of the resulting HPLC chromatogram may be determined. In certain embodiments, the weight ratio of the amount of an impurity to the amount of a desired substance is the ratio of the peak area of the impurity to the peak area of the desired substance.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. A “patient” refers to a human subject in need of treatment of a disease.

The terms “administer,” “administering,” or “administration” refers to implanting, absorbing, ingesting, injecting, inhaling, or otherwise introducing a compound, or a salt, co-crystal, amorphous form, or crystalline form thereof, or a pharmaceutical composition thereof, in or on a subject.

The terms “in combination” and “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a subject.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof). In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms. Treatment may also be continued after symptoms have resolved, for example, to delay or prevent recurrence.

The terms “prevention,” “prevent,” and “preventing” refer to administering a medicament (e.g., compound 109-3, or a salt, co-crystal, amorphous form, crystalline form, or pharmaceutical composition thereof) beforehand to avert or forestall the appearance of one or more symptoms of a disease or disorder. The person of ordinary skill in the medical art recognizes that the terms “prevention,” “prevent,” and “preventing” are not absolute terms. In the medical art these terms are understood to refer to the prophylactic administration of a medicament to substantially diminish the likelihood or seriousness of a condition, or symptom of the condition, and this is the sense intended in this disclosure.

The terms “condition,” “disease,” and “disorder” are used interchangeably.

An “effective amount” of a salt, co-crystal, amorphous form, or crystalline form described herein refers to an amount sufficient to elicit the desired biological response, e.g., treating a condition. The effective amount of a salt, co-crystal, amorphous form, or crystalline form described herein may vary depending on such factors as the desired biological endpoint, the pharmacokinetics of the salt, co-crystal, amorphous form, or crystalline form, the condition being treated, the mode of administration, and the age and health of the subject. An effective amount encompasses therapeutic and prophylactic treatment. For example, in treating a CARM1-mediated disorder, an effective amount of a salt, co-crystal, amorphous form, or crystalline form described herein may provide a therapeutic and/or prophylactic benefit in the treatment and/or prevention of the CARM1-mediated disorder or to delay or minimize one or more symptoms associated with the CARM1-mediated disorder.

A “therapeutically effective amount” of a salt, co-crystal, amorphous form, or crystalline form described herein is an amount sufficient to provide a therapeutic benefit in the treatment of a condition (e.g., a CARM1-mediated disorder) or to delay or minimize one or more symptoms associated with the condition. A therapeutically effective amount of a compound, or a salt, co-crystal, amorphous form, or crystalline form thereof, or a pharmaceutical composition thereof, means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a salt, co-crystal, amorphous form, or crystalline form described herein is an amount sufficient to prevent a condition (e.g., a CARM1-mediated disorder), or one or more symptoms associated with the condition or prevent its recurrence. A prophylactically effective amount of a compound, or a salt, co-crystal, amorphous form, or crystalline form thereof, or a pharmaceutical composition thereof, means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The term “methyltransferase” represents transferase class enzymes that are able to transfer a methyl group from a donor molecule to an acceptor molecule, e.g., an amino acid residue of a protein or a nucleic base of a DNA molecule. Methytransferases typically use a reactive methyl group bound to sulfur in S-adenosyl methionine (SAM) as the methyl donor. In some embodiments, a methyltransferase described herein is a protein methyltransferase. In some embodiments, a methyltransferase described herein is a histone methyltransferase. Histone methyltransferases (HMT) are histone-modifying enzymes, (including histone-lysine N-methyltransferase and histone-arginine N-methyltransferase), that catalyze the transfer of one or more methyl groups to lysine and/or arginine residues of histone proteins. In certain embodiments, a methyltransferase described herein is a histone-arginine N-methyltransferase.

Solid Forms

Compound 109-3 and forms of compound 109-3 associated with gentisic acid have been found to exist in a variety of forms, such as salts, co-crystals, amorphous forms, and crystalline forms as described herein.

Different solid forms of a compound (e.g., compound 109-3), salt, or co-crystal thereof typically differ in their physical and/or chemical properties due to the arrangement of the molecules in the solid form (e.g., the arrangement of the molecule in a crystal lattice). The different solid forms may even result in different pharmacokinetic and/or pharmacodynamic properties. The salts, co-crystals, amorphous forms, and crystalline forms may improve one or more physical, chemical, pharmacokinetic, and/or pharmacodynamic characteristics (e.g., increased solubility (e.g., aqueous solubility); increased permeability; increased stability; increased ease of formulation, storage, transportation, and/or administration; decreased costs of formation, transportation, storage, and/or administration; increased adsorption; modified distribution; increased bioavailability; increased or decreased metabolism; increased or decreased excretion; increased potency; increased efficacy; decreased toxicity; decreased frequency and/or severity of side effects; and/or increased patient compliance), compared with compound 109-3, an amorphous form of compound 109-3, an amorphous form of a salt of compound 109-3, a different crystalline form of compound 109-3, a different crystalline form of a salt of compound 109-3, and/or a different co-crystal comprising compound 109-3 and an acid.

Different solid forms of a compound (e.g., compound 109-3) can be typically distinguished by X-ray diffraction, in particular X-ray powder diffraction (XRPD, obtained by, e.g., a method described herein), and by other methods, such as, differential scanning calorimetry (DSC, e.g., modulated DSC (mDSC); obtained by, e.g., a method described herein), thermal gravimetric analysis (TGA, obtained by, e.g., a method described herein), and/or solubility (e.g., thermodynamic solubility).

(i) Form A

Compound 109-3 may be in an amorphous form. In some embodiments, the present disclosure provides amorphous form A (Form A) of compound 109-3. In certain embodiments, Form A is substantially not a salt (e.g., salt formed between compound 109-3 and an acid) or co-crystal (e.g., co-crystal comprising compound 109-3 and an acid). In some embodiments, Form A is obtained by precipitation of compound 109-3 from methanol, acetonitrile, acetone, or tetrahydrofuran (THF). In some embodiments, Form A is a solvate (e.g., stoichiometric solvate or non-stoichiometric solvate). In some embodiments, Form A is a hydrate, methanolate, acetonitrile solvate, acetone solvate, or THF solvate. In some embodiments, Form A does not include a solvent. In some embodiments, Form A is substantially anhydrous.

In certain embodiments, Form A is substantially free of impurities. In certain embodiments, Form A is at least 95%, at least 96%, at least 97%, at least 98%, at least 99%, or at least 99.5% by weight free of impurities. In certain embodiments, Form A is substantially free of crystalline forms of compound 109-3. In certain embodiments, Form A is substantially free of salts of compound 109-3. In certain embodiments, Form A is substantially free of co-crystals of compound 109-3 (e.g., co-crystals formed by compound 109-3 and an acid). In certain embodiments, Form A is substantially free of solvent (e.g., water).

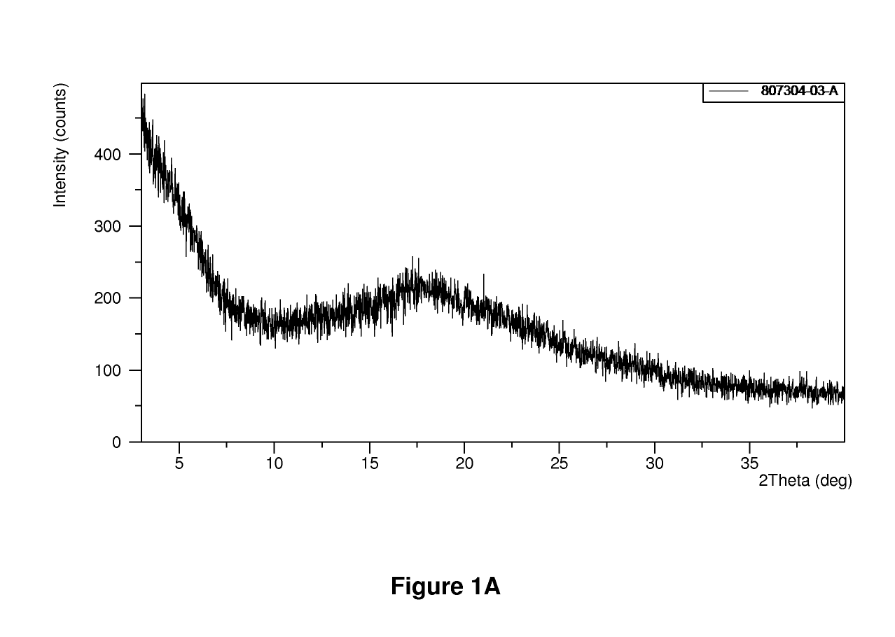

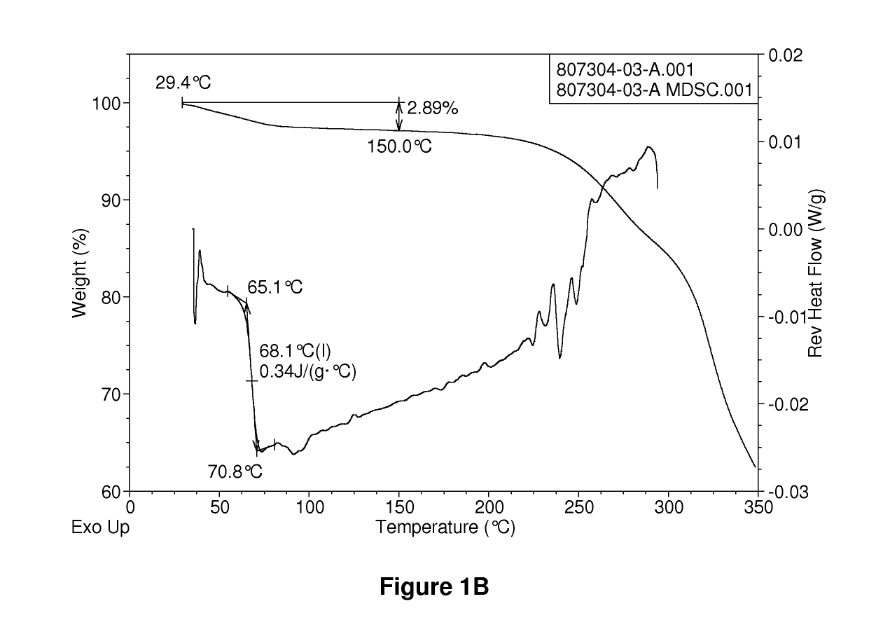

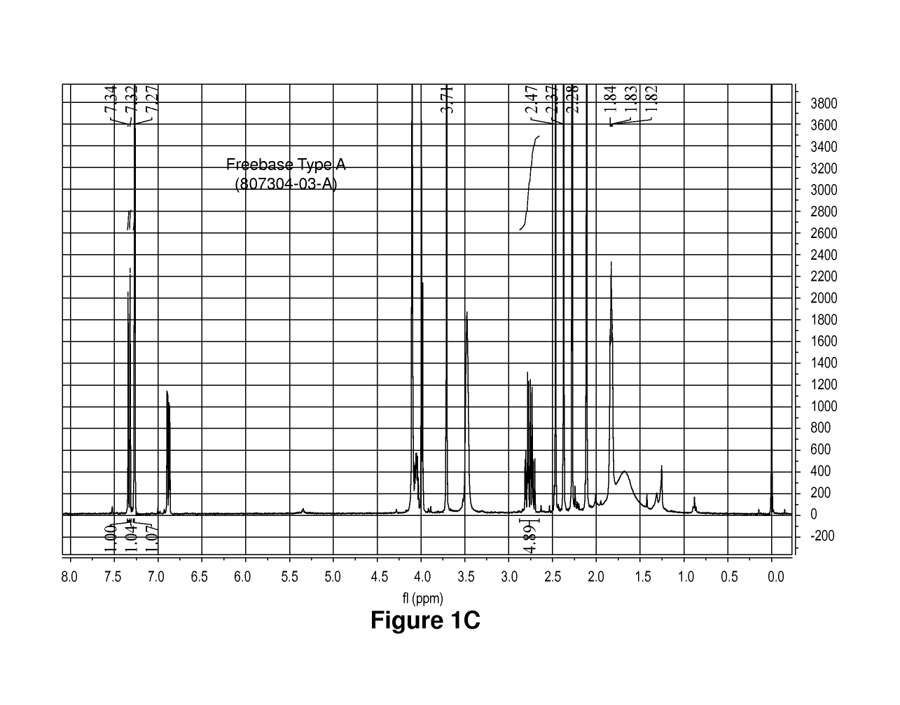

Form A can be characterized by one or more of the characteristics described herein, including, but not limited to, X-ray powder diffraction (XRPD) pattern, differential scanning calorimetry (DSC) thermogram, thermal gravimetric analysis (TGA) thermogram, and thermodynamic solubility. In some embodiments, Form A is characterized by an XRPD pattern substantially similar to the one depicted in FIG. 1A.

In some embodiments, Form A has a DSC thermogram substantially similar to the one depicted in FIG. 1B. In some embodiments, Form A is characterized by a DSC thermogram comprising an endotherm comprising a glass-transition temperature (T_{g}) of 65.1±2° C. In some embodiments, Form A is characterized in that it has a DSC thermogram comprising an endotherm comprising a midpoint temperature of 68.1±2° C. In some embodiments, Form A is characterized in that it has a DSC thermogram comprising an endotherm comprising a peak temperature (Tₘₐₓ) of 70.8±2° C. In some embodiments, Form A is characterized in that it has a DSC thermogram comprising a specific heat (Cₚ) of about 0.34 J/(g·° C.).

In some embodiments, Form A is characterized in that it has a TGA thermogram substantially similar to the one depicted in FIG. 1B. In certain embodiments, Form A is characterized in that it has a TGA thermogram comprising a weight loss of about 2.9% up to 150° C.

In certain embodiments, Form A is characterized in that it has (1) an XRPD pattern described herein for Form A and (2) a DSC thermogram comprising an endotherm comprising a T_{g} described herein for Form A. In certain embodiments, Form A is characterized in that it has (1) an XRPD pattern described herein for Form A and (2) a DSC thermogram comprising an endotherm comprising a midpoint temperature described herein for Form A. In certain embodiments, Form A is characterized in that it has (1) an XRPD pattern described herein for Form A and (2) a DSC thermogram comprising an endotherm comprising a Tₘₐₓ described herein for Form A.

In some embodiments, Form A is characterized in that it has a proton nuclear magnetic resonance (¹H-NMR) spectrum substantially similar to the one depicted in FIG. 1C.

In some embodiments, Form A is characterized in that it has one or more thermodynamic solubilities as shown in Table 1.

TABLE 1
Exemplary thermodynamic solubilities
of Form A at room temperature.

| | Solubility |
|---|---|
| Solvent | (mg/mL) |
| MeOH | >38.0 |
| EtOH | >40.0 |
| Isopropanol | >36.0 |
| Acetonitrile | >42.0 |
| Acetone | >48.0 |
| Methyl isobutyl | >34.0 |
| ketone | |
| EtOAc | >50.0 |
| Isopropyl acetate | >36.0 |
| Methyl-tert- | <1.1 |
| butyl ether | |
| THF | >36.0 |
| Acetic acid | >44.0 |
| 2-Methyl- | >50.0 |
| tetrahydrofuran | |
| 1,4-Dioxane | >40.0 |
| N-methyl-2- | >44.0 |
| pyrrolidone | |
| Dimethyl sulfoxide | >38.0 |
| CHCl₃ | >38.0 |
| Toluene | >38.0 |
| n-Heptane | <1.1 |
| Dimethylacetamide | >42.0 |
| H₂O | <1.1 |
| Dichloromethane | >36.0 |
| n-Heptane/THF | >40.0 |
| (1:3 by volume) | |
| n-Heptane/THF | >42.0 |
| (1:2 by volume) | |
| n-Heptane/THF | between 14.0 and |
| (1:1 by volume) | 21.0 |
| n-Heptane/THF | |
| (2:1 by volume) | between 2.6 and 3.0 |
| n-Heptane/THF | <1.6 |
| (4:1 by volume) | |

In some embodiments, Form A is stable for at least 1 month, at least 2 months, at least 4 months, at least 6 months, at least 12 months, at least 18 months, at least 24 months, or at least 3 years at 25° C. and about 60% relative humidity. In some embodiments, Form A has substantially the same XRPD pattern post storage for at least 1 month, at least 2 months, at least 4 months, at least 6 months, at least 12 months, at least 18 months, at least 24 months, or at least 3 years at 25° C. and about 60% relative humidity.

In some embodiments, Form A is stable for at least 1 month, at least 2 months, at least 4 months, at least 6 months, at least 8 months, at least 10 months, at least 12 months, at least 18 months, or at least 24 months at 40° C. and about 75% relative humidity. In some embodiments, Form A has substantially the same XRPD pattern post storage for at least 1 month, at least 2 months, at least 4 months, at least 6 months, at least 8 months, at least 10 months, at least 12 months, at least 18 months, or at least 24 months at 40° C. and about 75% relative humidity.

(ii) Gentisate Salts, Form G-A, Form G-B, and Co-crystals Comprising Gentisic Acid

In some embodiments, described herein are gentisate salts of compound 109-3. In certain embodiments, the molar ratio of gentisic acid to compound 109-3 in a gentisate salt of compound 109-3 is about 1:1.

In certain embodiments, a gentisate salt of compound 109-3 is substantially free of impurities. In certain embodiments, a gentisate salt of compound 109-3 is at least 95%, at least 96%, at least 97%, at least 98%, at least 99%, or at least 99.5% by weight free of impurities (e.g., other salts of compound 109-3). In some embodiments, a gentisate salt of compound 109-3 is a solvate (e.g., stoichiometric solvate or non-stoichiometric solvate). In some embodiments, a gentisate salt of compound 109-3 is a methanolate, acetonitrile solvate, acetone solvate, THF solvate, or n-heptane solvate. In certain embodiments, a gentisate salt of compound 109-3 is a hydrate. In some embodiments, a gentisate salt of compound 109-3 does not include a solvent. In some embodiments, a gentisate salt of compound 109-3 is substantially anhydrous.

In another aspect, the present disclosure provides co-crystals comprising compound 109-3 and gentisic acid. In certain embodiments, the molar ratio of gentisic acid to compound 109-3 in a co-crystal comprising compound 109-3 and gentisic acid is about 1:1.

In certain embodiments, a co-crystal comprising compound 109-3 and gentisic acid is substantially free of impurities. In certain embodiments, a co-crystal comprising compound 109-3 and gentisic acid is at least 95%, at least 96%, at least 97%, at least 98%, at least 99%, or at least 99.5% by weight free of impurities (e.g., other salts or co-crystals of compound 109-3). In some embodiments, a co-crystal comprising compound 109-3 and gentisic acid is a solvate (e.g., stoichiometric solvate or non-stoichiometric solvate). In some embodiments, a co-crystal comprising compound 109-3 and gentisic acid is a methanolate, acetonitrile solvate, acetone solvate, THF solvate, or n-heptane solvate. In some embodiments, a co-crystal comprising compound 109-3 and gentisic acid is a hydrate. In some embodiments, a co-crystal comprising compound 109-3 and gentisic acid does not include a solvent. In some embodiments, a co-crystal comprising compound 109-3 and gentisic acid is substantially anhydrous.

In certain embodiments, the present disclosure provides crystalline form G-A (Form G-A) of compound 109-3, wherein Form G-A comprises gentisic acid. In some embodiments, Form G-A is a gentisate salt of compound 109-3. In some embodiments, Form G-A is a co-crystal of compound 109-3 and gentisic acid. In some embodiments, the molar ratio of gentisic acid to compound 109-3 in Form G-A is about 1:1.

In some embodiments, Form G-A is obtained by recrystallization of a gentisate salt (e.g., monogentisate salt) of compound 109-3 from methanol, acetonitrile, acetone, or THF. In some embodiments, Form G-A is obtained by recrystallization of compound 109-3 from a solution of gentisic acid in methanol, acetonitrile, acetone, or THF (e.g., a solution containing one or more equivalents of gentisic acid, where the amount of compound 109-3 is one equivalent). In some embodiments, Form G-A is a solvate (e.g., stoichiometric solvate or non-stoichiometric solvate). In some embodiments, Form G-A is a methanolate, acetonitrile solvate, acetone solvate, or THF solvate. In some embodiments, Form G-A is a hydrate (e.g., monohydrate). In some embodiments, Form G-A does not include a solvent. In some embodiments, Form G-A is substantially anhydrous.

In certain embodiments, Form G-A is substantially free of impurities. In certain embodiments, Form G-A is at least 95%, at least 96%, at least 97%, at least 98%, at least 99%, or at least 99.5% by weight free of impurities. In certain embodiments, Form G-A is substantially free of amorphous forms of compound 109-3 or amorphous forms of a salt of compound 109-3. In certain embodiments, Form G-A is substantially free of other crystalline forms of compound 109-3 or other crystalline forms of a salt of compound 109-3. In certain embodiments, Form G-A is substantially free of other salts of compound 109-3. In certain embodiments, Form G-A is substantially free of solvents (e.g., water, methanol, acetonitrile, acetone, or THF).

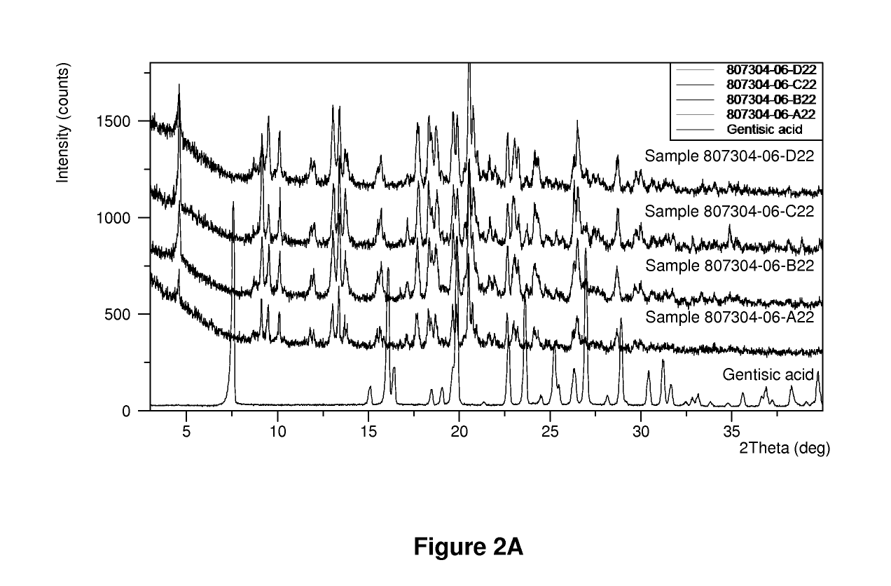

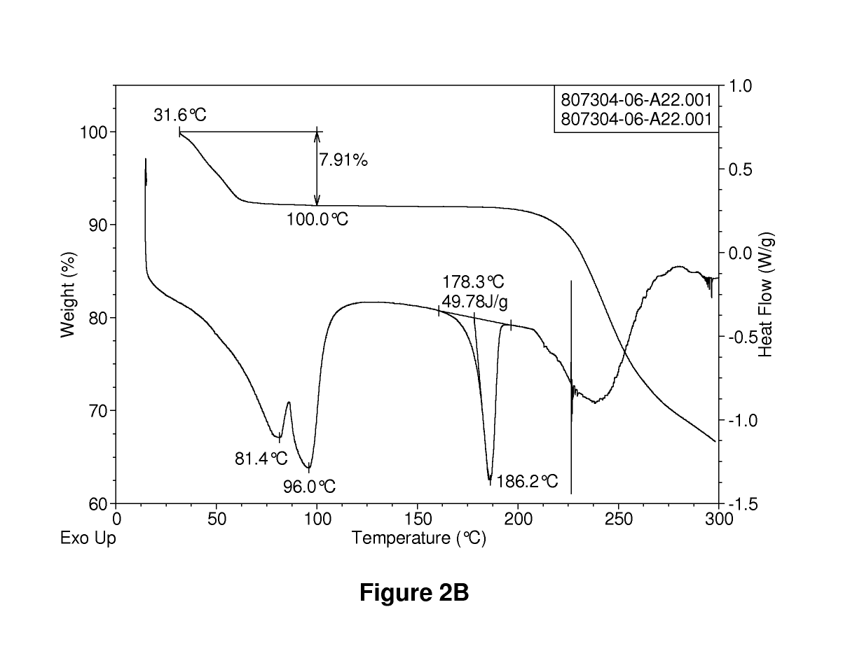

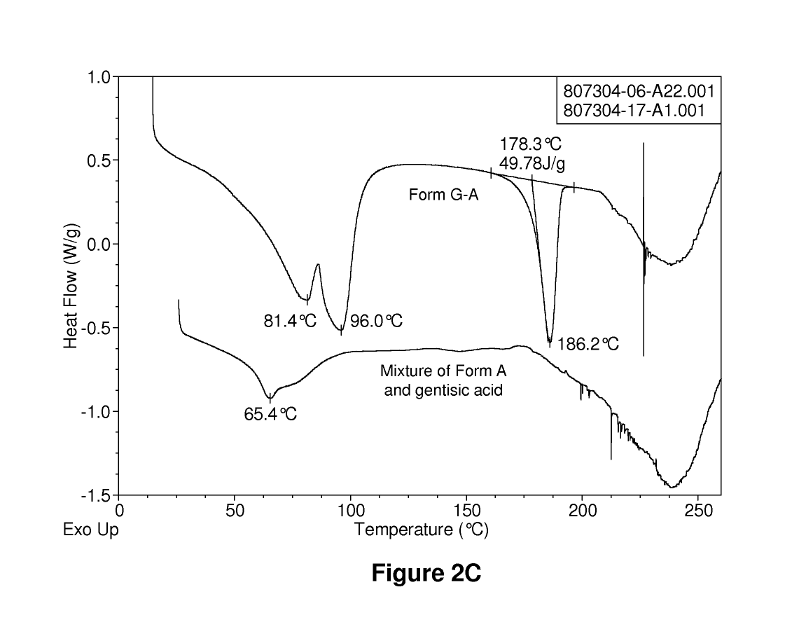

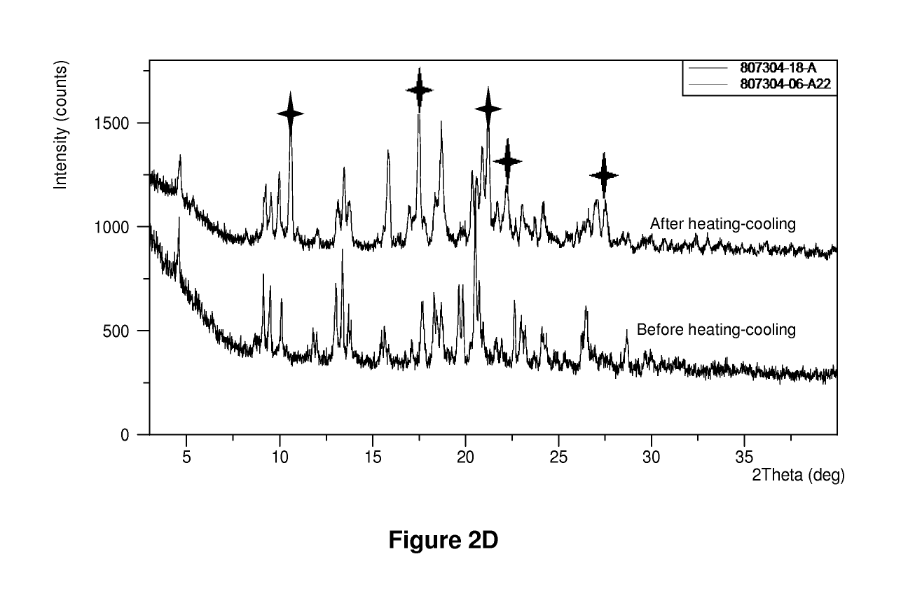

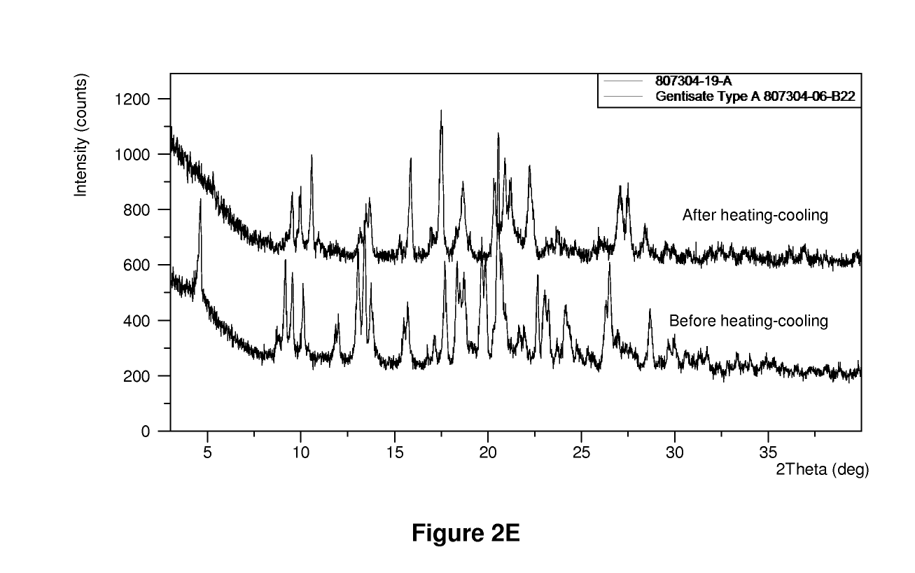

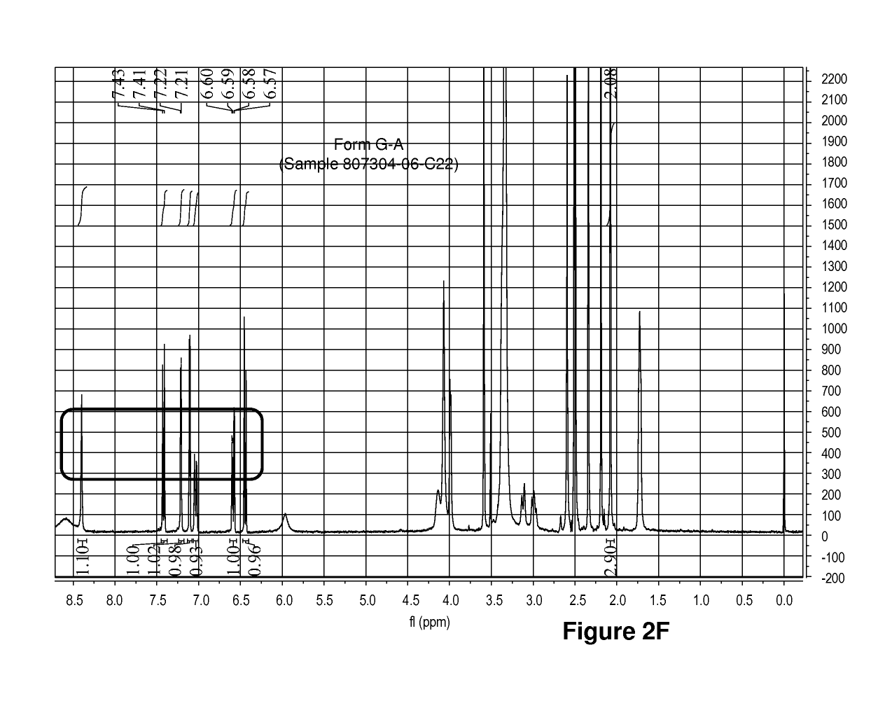

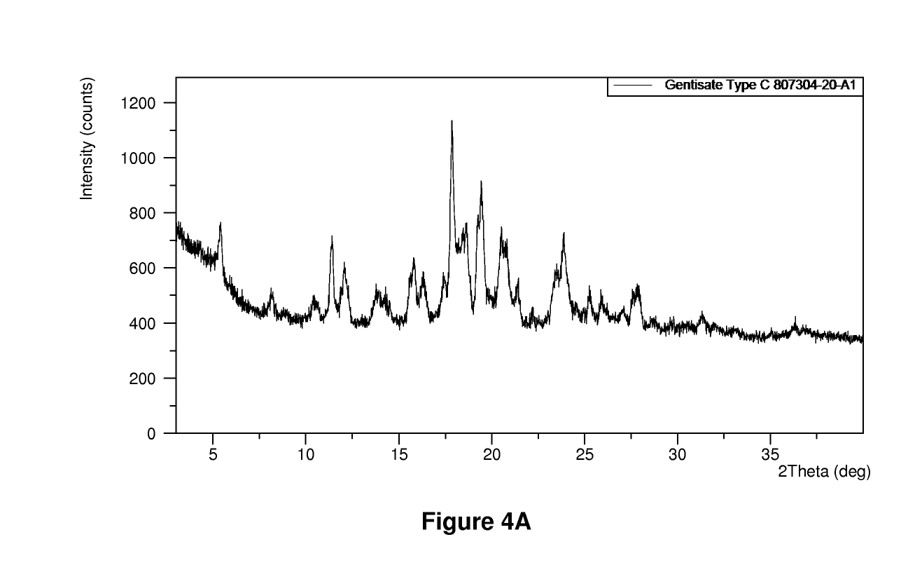

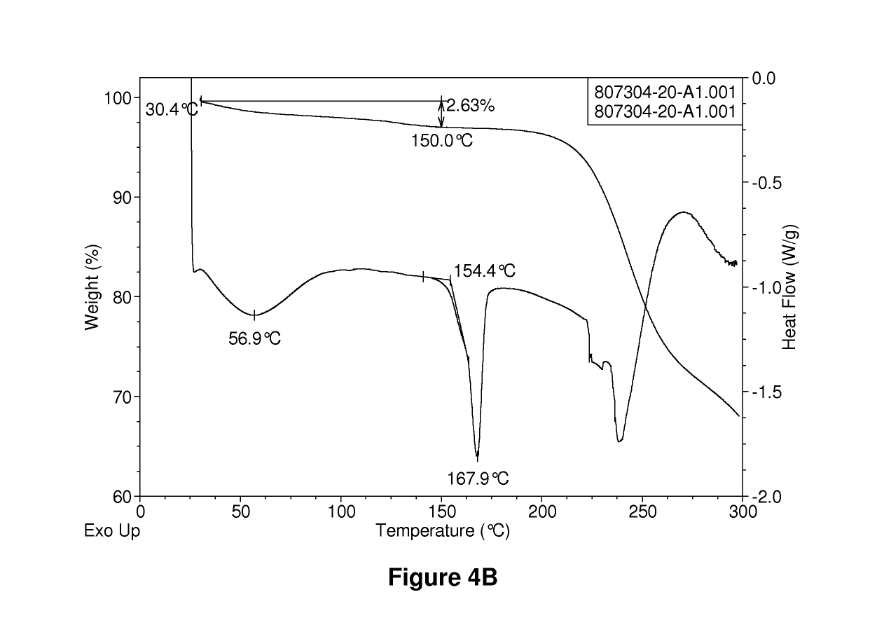

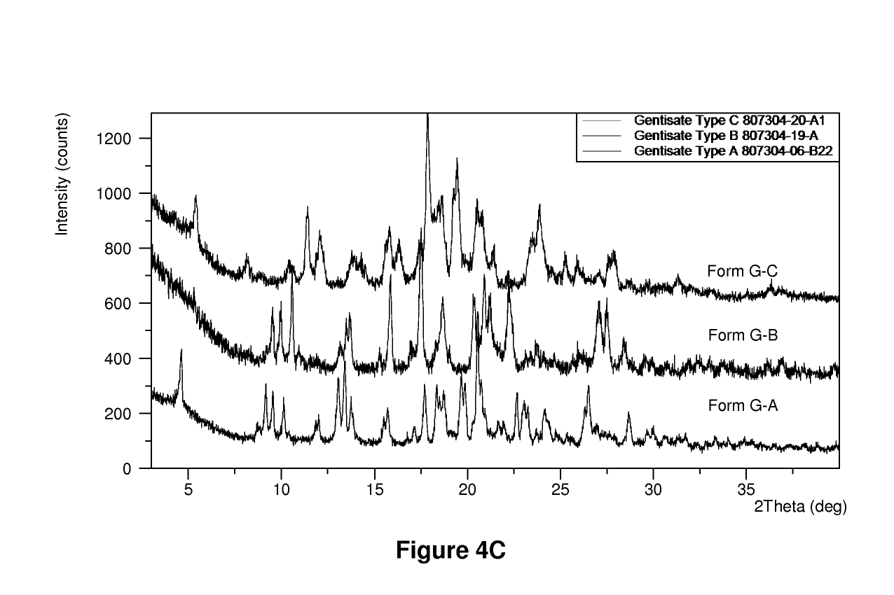

Form G-A can be characterized by one or more of the characteristics described herein, including, but not limited to, XRPD pattern, DSC thermogram, TGA thermogram, and proton nuclear magnetic resonance (¹H-NMR). In some embodiments, Form G-A is characterized by an XRPD pattern substantially similar to the one depicted in FIG. 2A (e.g., Sample 807304-06-A22) or FIG. 4C (e.g., Sample 807304-06-B22). In some embodiments, Form G-A is characterized by an XRPD pattern comprising one or more, two or more, three or more, four or more, five or more, six or more, seven or more, eight or more, or nine characteristic peaks, each of which independently has an angle 2-theta value shown in Table 2. In some embodiments, Form G-A is characterized by an XRPD pattern comprising a characteristic peak having an angle 2-theta value the same as the angle 2-theta value of Peak Number 1 shown in Table 2. In some embodiments, Form G-A is characterized by an XRPD pattern comprising two characteristic peaks having angle 2-theta values the same as the angle 2-theta values of Peak Numbers 1 and 2 shown in Table 2, respectively. In some embodiments, Form G-A is characterized by an XRPD pattern comprising three characteristic peaks having angle 2-theta values the same as the angle 2-theta values of Peak Numbers 1 to 3 shown in Table 2, respectively. In some embodiments, Form G-A is characterized by an XRPD pattern comprising four characteristic peaks having angle 2-theta values the same as the angle 2-theta values of Peak Numbers 1 to 4 shown in Table 2, respectively. In some embodiments, Form G-A is characterized by an XRPD pattern where the angle 2-theta value of the most intense peak, each of the first to second most intense peaks, each of the first to third most intense peaks, each of the first to fourth most intense peaks, each of the first to fifth most intense peaks, each of the first to sixth most intense peaks, each of the first to seventh most intense peaks, each of the first to eighth most intense peaks, or each of the first to ninth most intense peaks independently is the same as an angle 2-theta value shown in Table 2.

TABLE 2
Exemplary characteristic peaks from
the X-ray powder diffraction pattern.

| Peak | Angle | Relative |
|---|---|---|
| Number | 2-theta (°) | intensity (%) |
| 1 | 20.51 ± 0.2 | 100.0 |
| 2 | 13.37 ± 0.2 | 57.2 |
| 3 | 9.13 ± 0.2 | 45.15 |
| 4 | 19.64 ± 0.2 | 43.45 |
| 5 | 13.04 ± 0.2 | 43.06 |
| 6 | 19.86 ± 0.2 | 39.22 |
| 7 | 18.30 ± 0.2 | 36.85 |
| 8 | 9.49 ± 0.2 | 36.51 |
| 9 | 18.68 ± 0.2 | 33.95 |

In some embodiments, Form G-A has a DSC thermogram substantially similar to the one depicted in FIG. 2B. In some embodiments, Form G-A is characterized in that it has a DSC thermogram comprising an endotherm comprising an onset temperature (Tₘ) of 178.3±2° C. In some embodiments, Form G-A is characterized in that it has a DSC thermogram comprising an endotherm comprising a Tₘₐₓ of 186.2±2° C. In some embodiments, Form G-A is characterized in that it has a DSC thermogram further comprising another endotherm comprising a Tₘₐₓ of 96.0±2° C. In some embodiments, Form G-A is characterized in that it has a DSC thermogram further comprising another endotherm comprising a Tₘₐₓ of 81.4±2° C. In some embodiments, Form G-A is characterized in that it has a DSC thermogram comprising a ΔH of about 49.78 J/g.

In some embodiments, Form G-A is characterized in that it has a TGA thermogram substantially similar to the one depicted in FIG. 2B. In certain embodiments, Form G-A is characterized in that it has a TGA thermogram comprising a weight loss of about 7.9% up to 100° C.

In certain embodiments, Form G-A is characterized in that it has (1) an XRPD pattern described herein for Form G-A and (2) a DSC thermogram comprising an endotherm comprising a Tₘ described herein for Form G-A. In certain embodiments, Form G-A is characterized in that it has (1) an XRPD pattern described herein for Form G-A and (2) a DSC thermogram comprising an endotherm comprising a Tₘₐₓ described herein for Form G-A.

In some embodiments, Form G-A is characterized in that it has a ¹H-NMR spectrum substantially similar to the one depicted in FIG. 2F.

In some embodiments, Form G-A is stable for at least 1 month, at least 2 months, at least 4 months, at least 6 months, at least 12 months, at least 18 months, at least 24 months, or at least 3 years at 25° C. and about 60% relative humidity. In some embodiments, Form G-A has substantially the same XRPD pattern post storage for at least 1 month, at least 2 months, at least 4 months, at least 6 months, at least 12 months, at least 18 months, at least 24 months, or at least 3 years at 25° C. and about 60% relative humidity.

In some embodiments, Form G-A is stable for at least 1 month, at least 2 months, at least 4 months, at least 6 months, at least 8 months, at least 10 months, at least 12 months, at least 18 months, or at least 24 months at 40° C. and about 75% relative humidity. In some embodiments, Form G-A has substantially the same XRPD pattern post storage for at least 1 month, at least 2 months, at least 4 months, at least 6 months, at least 8 months, at least 10 months, at least 12 months, at least 18 months, or at least 24 months at 40° C. and about 75% relative humidity.

In some embodiments, the present disclosure provides crystalline form G-B (Form G-B) of compound 109-3, wherein Form G-B comprises gentisic acid. In some embodiments, Form G-B is a gentisate salt of compound 109-3. In some embodiments, Form G-B is a co-crystal of compound 109-3 and gentisic acid. In some embodiments, the molar ratio of gentisic acid to compound 109-3 in Form G-B is about 1:1.

In some embodiments, Form G-B is obtained by heating Form G-A to a first temperature (e.g., about 80° C., about 90° C., about 100° C., about 110° C., or about 120° C., such as about 100° C.); optionally substantially maintaining the temperature of Form G-A at the first temperature for at least about 1 minutes, at least about 10 minutes, at least about 1 hour, at least about 6 hours, or at least about 24 hours; and then cooling it to room temperature (e.g., about 25° C.). In some embodiments, Form G-B does not include a solvent. In some embodiments, Form G-B is substantially anhydrous.

In some embodiments, Form G-B is a solvate (e.g., stoichiometric solvate or non-stoichiometric solvate). In some embodiments, Form G-B is a methanolate, acetonitrile solvate, acetone solvate, or THF solvate. In some embodiments, Form G-B is a hydrate (e.g., monohydrate). In some embodiments, Form G-B does not include a solvent. In some embodiments, Form G-B is substantially anhydrous.

In certain embodiments, Form G-B is substantially free of impurities. In certain embodiments, Form G-B is at least 95%, at least 96%, at least 97%, at least 98%, at least 99%, or at least 99.5% by weight free of impurities. In certain embodiments, Form G-B is substantially free of amorphous forms of compound 109-3 or amorphous forms of a salt of compound 109-3. In certain embodiments, Form G-B is substantially free of other crystalline forms of compound 109-3 or other crystalline forms of a salt of compound 109-3. In certain embodiments, Form G-B is substantially free of other salts of compound 109-3. In certain embodiments, Form G-B is substantially free of solvents (e.g., water, methanol, acetonitrile, acetone, or THF).

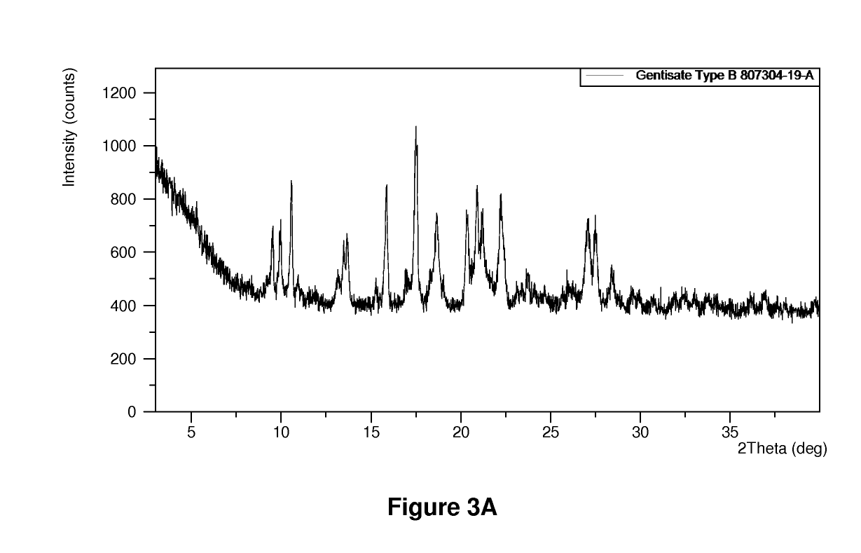

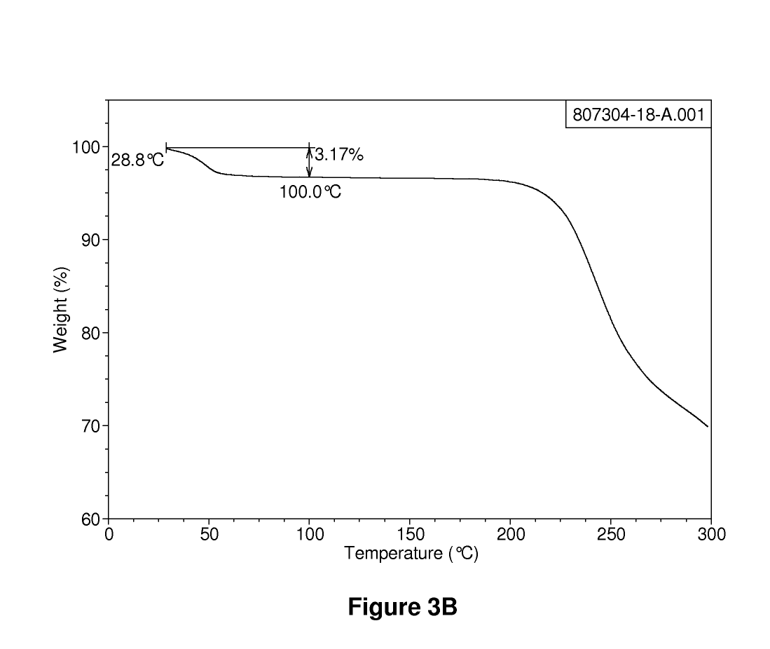

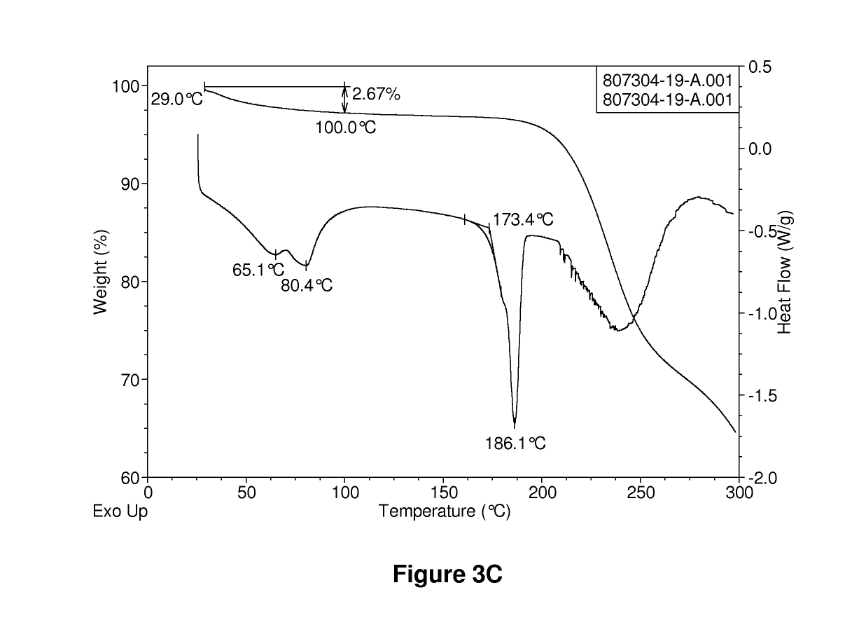

Form G-B can be characterized by one or more of the characteristics described herein, including, but not limited to, XRPD pattern, DSC thermogram, and TGA thermogram. In some embodiments, Form G-B is characterized by an XRPD pattern substantially similar to the one depicted in FIG. 3A or FIG. 4C (e.g., Sample 807304-19-A). In some embodiments, Form G-B is characterized by an XRPD pattern comprising one or more, two or more, three or more, four or more, five or more, six or more, seven or more, eight or more, or nine characteristic peaks, each of which independently has an angle 2-theta value shown in Table 3. In some embodiments, Form G-B is characterized by an XRPD pattern comprising a characteristic peak having an angle 2-theta value substantially the same as the angle 2-theta value of Peak Number 1 shown in Table 3. In some embodiments, Form G-B is characterized by an XRPD pattern comprising two characteristic peaks having angle 2-theta values substantially the same as the angle 2-theta values of Peak Numbers 1 and 2 shown in Table 3, respectively. In some embodiments, Form G-B is characterized by an XRPD pattern comprising three characteristic peaks having angle 2-theta values substantially the same as the angle 2-theta values of Peak Numbers 1 to 3 shown in Table 3, respectively. In some embodiments, Form G-B is characterized by an XRPD pattern comprising four characteristic peaks having angle 2-theta values substantially the same as the angle 2-theta values of Peak Numbers 1 to 4 shown in Table 3, respectively. In some embodiments, Form G-B is characterized by an XRPD pattern where the angle 2-theta value of the most intense peak, each of the first to second most intense peaks, each of the first to third most intense peaks, each of the first to fourth most intense peaks, each of the first to fifth most intense peaks, each of the first to sixth most intense peaks, each of the first to seventh most intense peaks, each of the first to eighth most intense peaks, or each of the first to ninth most intense peaks independently is the same as an angle 2-theta value shown in Table 3.

TABLE 3
Exemplary characteristic peaks from
the X-ray powder diffraction pattern.

| Peak | Angle | Relative |
|---|---|---|
| Number | 2-theta (°) | intensity (%) |
| 1 | 17.53 ± 0.2 | 100.0 |
| 2 | 15.87 ± 0.2 | 72.1 |
| 3 | 10.57 ± 0.2 | 70.4 |
| 4 | 20.92 ± 0.2 | 68.9 |
| 5 | 22.22 ± 0.2 | 63.3 |
| 6 | 18.67 ± 0.2 | 54.6 |
| 7 | 21.21 ± 0.2 | 53.2 |
| 8 | 20.34 ± 0.2 | 52.7 |
| 9 | 27.06 ± 0.2 | 47.2 |

In certain embodiments, Form G-B is characterized by a DSC thermogram substantially similar to the one depicted in FIG. 3C. In certain embodiments, Form G-B is characterized by a DSC thermogram comprising an endotherm comprising a Tₘ of 173.4±2° C. In certain embodiments, Form G-B is characterized by a DSC thermogram comprising an endotherm comprising a Tₘₐₓ of 186.1±2° C. In certain embodiments, Form G-B is characterized by a DSC thermogram further comprising another endotherm comprising a Tₘₐₓ of 80.4±2° C. In certain embodiments, Form G-B is characterized by a DSC thermogram further comprising another endotherm comprising a Tₘₐₓ of 65.1±2° C.

In certain embodiments, Form G-B is characterized in that it has (1) an XRPD pattern described herein for Form G-B and (2) a DSC thermogram comprising an endotherm comprising a Tₘ described herein for Form G-B. In certain embodiments, Form G-B is characterized in that it has (1) an XRPD pattern described herein for Form G-B and (2) a DSC thermogram comprising an endotherm comprising a Tₘₐₓ described herein for Form G-B.

In some embodiments, Form G-B is characterized in that it has a TGA thermogram substantially similar to the one depicted in FIG. 3B. In certain embodiments, Form G-B is characterized in that it has a TGA thermogram comprising a weight loss of about 3.2% up to 100° C.

In some embodiments, Form G-B is characterized in that it has a TGA thermogram substantially similar to the one depicted in FIG. 3C. In certain embodiments, Form G-B is characterized in that it has a TGA thermogram comprising a weight loss of about 2.7% up to 100° C.

In some embodiments, Form G-B is stable for at least 1 month, at least 2 months, at least 4 months, at least 6 months, at least 12 months, at least 18 months, at least 24 months, or at least 3 years at 25° C. and about 60% relative humidity. In some embodiments, Form G-B has substantially the same XRPD pattern post storage for at least 1 month, at least 2 months, at least 4 months, at least 6 months, at least 12 months, at least 18 months, at least 24 months, or at least 3 years at 25° C. and about 60% relative humidity.

In some embodiments, Form G-B is stable for at least 1 month, at least 2 months, at least 4 months, at least 6 months, at least 8 months, at least 10 months, at least 12 months, at least 18 months, or at least 24 months at 40° C. and about 75% relative humidity. In some embodiments, Form G-B has substantially the same XRPD pattern post storage for at least 1 month, at least 2 months, at least 4 months, at least 6 months, at least 8 months, at least 10 months, at least 12 months, at least 18 months, or at least 24 months at 40° C. and about 75% relative humidity.

In some embodiments, the present disclosure provides crystalline form G-C (Form G-C) of compound 109-3, wherein Form G-C comprises gentisic acid. In some embodiments, Form G-C is a gentisate salt of compound 109-3. In some embodiments, Form G-C is a co-crystal of compound 109-3 and gentisic acid. In some embodiments, the molar ratio of gentisic acid to compound 109-3 in Form G-C is about 1:1.

In some embodiments, Form G-C is obtained by recrystallization of a gentisate salt (e.g., monogentisate salt) of compound 109-3 from a mixture of n-heptane and acetone (e.g., at about 1:1 or about 1.5:1 ratio by volume) or a mixture of n-heptane and THF (e.g., at about 1:1 ratio by volume). In some embodiments, Form G-C is obtained by recrystallization of compound 109-3 from a solution of gentisic acid in a mixture of n-heptane of acetone or a mixture of n-heptane and THF (e.g., a solution containing one or more equivalents of gentisic acid, where the amount of compound 109-3 is one equivalent). In some embodiments, Form G-C is a solvate (e.g., stoichiometric solvate or non-stoichiometric solvate). In some embodiments, Form G-C is a n-heptane solvate, acetone solvate, or THF solvate. In some embodiments, Form G-C is a hydrate (e.g., monohydrate). In some embodiments, Form G-C does not include a solvent. In some embodiments, Form G-C is substantially anhydrous.

In certain embodiments, Form G-C is substantially free of impurities. In certain embodiments, Form G-C is at least 95%, at least 96%, at least 97%, at least 98%, at least 99%, or at least 99.5% by weight free of impurities. In certain embodiments, Form G-C is substantially free of amorphous forms of compound 109-3 or amorphous forms of a salt of compound 109-3. In certain embodiments, Form G-C is substantially free of other crystalline forms of compound 109-3 or other crystalline forms of a salt of compound 109-3. In certain embodiments, Form G-C is substantially free of other salts of compound 109-3. In certain embodiments, Form G-C is substantially free of solvents (e.g., water, n-heptane, acetone, or THF).

Form G-C can be characterized by one or more of the characteristics described herein, including, but not limited to, XRPD pattern, DSC thermogram, and TGA thermogram. In some embodiments, Form G-C is characterized by an XRPD pattern substantially similar to the one depicted in FIG. 4A or FIG. 4C (e.g., Sample 807304-20-A1). In some embodiments, Form G-C is characterized by an XRPD pattern comprising one or more, two or more, three or more, four or more, five or more, six or more, seven or more, eight or more, or nine characteristic peaks, each of which independently has an angle 2-theta value shown in Table 4. In some embodiments, Form G-C is characterized by an XRPD pattern comprising a characteristic peak having an angle 2-theta value substantially the same as the angle 2-theta value of Peak Number 1 shown in Table 4. In some embodiments, Form G-C is characterized by an XRPD pattern comprising two characteristic peaks having angle 2-theta values substantially the same as the angle 2-theta values of Peak Numbers 1 and 2 shown in Table 4, respectively. In some embodiments, Form G-C is characterized by an XRPD pattern comprising three characteristic peaks having angle 2-theta values substantially the same as the angle 2-theta values of Peak Numbers 1 to 3 shown in Table 4, respectively. In some embodiments, Form G-C is characterized by an XRPD pattern comprising four characteristic peaks having angle 2-theta values substantially the same as the angle 2-theta values of Peak Numbers 1 to 4 shown in Table 4, respectively. In some embodiments, Form G-C is characterized by an XRPD pattern where the angle 2-theta value of the most intense peak, each of the first to second most intense peaks, each of the first to third most intense peaks, each of the first to fourth most intense peaks, each of the first to fifth most intense peaks, each of the first to sixth most intense peaks, each of the first to seventh most intense peaks, each of the first to eighth most intense peaks, or each of the first to ninth most intense peaks independently is the same as an angle 2-theta value shown in Table 4.

TABLE 4
Exemplary characteristic peaks from
the X-ray powder diffraction pattern.

| Peak | Angle | Relative |
|---|---|---|
| Number | 2-theta (°) | intensity (%) |
| 1 | 17.86 ± 0.2 | 100.0 |
| 2 | 19.44 ± 0.2 | 66.0 |
| 3 | 19.24 ± 0.2 | 50.4 |
| 4 | 18.65 ± 0.2 | 47.8 |
| 5 | 20.49 ± 0.2 | 42.4 |
| 6 | 23.88 ± 0.2 | 42.2 |
| 7 | 11.43 ± 0.2 | 38.2 |
| 8 | 15.83 ± 0.2 | 30.0 |
| 9 | 23.45 ± 0.2 | 26.5 |

In some embodiments, Form G-C has a DSC thermogram substantially similar to the one depicted in FIG. 4B. In some embodiments, Form G-C is characterized in that it has a DSC thermogram comprising an endotherm comprising a Tₘ of 154.4±2° C. In some embodiments, Form G-C is characterized in that it has a DSC thermogram comprising an endotherm comprising a Tₘₐₓ of 167.9±2° C. In some embodiments, Form G-C is characterized in that it has a DSC thermogram further comprising another endotherm comprising a Tₘₐₓ of 56.9±2° C.

In certain embodiments, Form G-C is characterized in that it has (1) an XRPD pattern described herein for Form G-C and (2) a DSC thermogram comprising an endotherm comprising a Tₘ described herein for Form G-C. In certain embodiments, Form G-C is characterized in that it has (1) an XRPD pattern described herein for Form G-C and (2) a DSC thermogram comprising an endotherm comprising a Tₘₐₓ described herein for Form G-C.

In some embodiments, Form G-C is characterized in that it has a TGA thermogram substantially similar to the one depicted in FIG. 4B. In certain embodiments, Form G-C is characterized in that it has a TGA thermogram comprising a weight loss of about 2.6% up to 150° C.

In some embodiments, Form G-C is stable for at least 1 month, at least 2 months, at least 4 months, at least 6 months, at least 12 months, at least 18 months, at least 24 months, or at least 3 years at 25° C. and about 60% relative humidity. In some embodiments, Form G-C has substantially the same XRPD pattern post storage for at least 1 month, at least 2 months, at least 4 months, at least 6 months, at least 12 months, at least 18 months, at least 24 months, or at least 3 years at 25° C. and about 60% relative humidity.

In some embodiments, Form G-C is stable for at least 1 month, at least 2 months, at least 4 months, at least 6 months, at least 8 months, at least 10 months, at least 12 months, at least 18 months, or at least 24 months at 40° C. and about 75% relative humidity. In some embodiments, Form G-C has substantially the same XRPD pattern post storage for at least 1 month, at least 2 months, at least 4 months, at least 6 months, at least 8 months, at least 10 months, at least 12 months, at least 18 months, or at least 24 months at 40° C. and about 75% relative humidity.

Pharmaceutical Compositions, Kits, Methods of Treatment, and Uses

A salt, co-crystal, amorphous form, or crystalline form described herein may inhibit CARM1 (e.g., inhibit the activity of CARM1). In certain embodiments, a salt, co-crystal, amorphous form, or crystalline form described herein inhibits wild-type CARM1. In certain embodiments, a salt, co-crystal, amorphous form, or crystalline form described herein inhibits a mutant CARM1. In certain embodiments, a salt, co-crystal, amorphous form, or crystalline form described herein inhibits CARM1, e.g., as measured in an assay described herein. In certain embodiments, the CARM1 is from a human. In certain embodiments, a salt, co-crystal, amorphous form, or crystalline form described herein inhibits CARM1 at an IC₅₀ less than or equal to 10 μM. In certain embodiments, a salt, co-crystal, amorphous form, or crystalline form described herein inhibits CARM1 at an IC₅₀ less than or equal to 1 μM. In certain embodiments, a salt, co-crystal, amorphous form, or crystalline form described herein inhibits CARM1 at an IC₅₀ less than or equal to 0.1 μM. In certain embodiments, a salt, co-crystal, amorphous form, or crystalline form described herein inhibits CARM1 in a cell at an EC₅₀ less than or equal to 10 μM. In certain embodiments, a salt, co-crystal, amorphous form, or crystalline form described herein inhibits CARM1 in a cell at an EC₅₀ less than or equal to 1 μM. In certain embodiments, a salt, co-crystal, amorphous form, or crystalline form described herein inhibits CARM1 in a cell at an EC₅₀ less than or equal to 0.1 μM. In certain embodiments, a salt, co-crystal, amorphous form, or crystalline form described herein inhibits cell proliferation at an EC₅₀ less than or equal to 10 μM. In certain embodiments, a salt, co-crystal, amorphous form, or crystalline form described herein inhibits cell proliferation at an EC₅₀ less than or equal to 1 μM. In certain embodiments, a salt, co-crystal, amorphous form, or crystalline form described herein inhibits cell proliferation at an EC₅₀ less than or equal to 0.1 μM. In some embodiments, a salt, co-crystal, amorphous form, or crystalline form described herein is selective for CARM1 over other methyltransferases. In certain embodiments, a salt, co-crystal, amorphous form, or crystalline form described herein is at least about 10-fold selective, at least about 20-fold selective, at least about 30-fold selective, at least about 40-fold selective, at least about 50-fold selective, at least about 60-fold selective, at least about 70-fold selective, at least about 80-fold selective, at least about 90-fold selective, or at least about 100-fold selective for PRMT1 relative to one or more other methyltransferases.

It will be understood by one of ordinary skill in the art that the CARM1 can be wild-type CARM1, or any mutant or variant of CARM1.

The present disclosure provides pharmaceutical compositions comprising a salt, co-crystal, amorphous form, or crystalline form described herein, and optionally a pharmaceutically acceptable excipient. The salts, co-crystals, amorphous forms, and crystalline forms may be present in various forms, such as amorphous, hydrates, solvates, or polymorphs. In certain embodiments, a provided composition comprises two or more salts, co-crystals, amorphous forms, and/or crystalline forms described herein. In certain embodiments, a salt, co-crystal, amorphous form, or crystalline form described herein is provided in an effective amount in the pharmaceutical composition. In certain embodiments, the effective amount is a therapeutically effective amount. In certain embodiments, the effective amount is an amount effective for inhibiting CARM1. In certain embodiments, the effective amount is an amount effective for treating a CARM1-mediated disorder. In certain embodiments, the effective amount is a prophylactically effective amount. In certain embodiments, the effective amount is an amount effective to prevent a CARM1-mediated disorder.

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing a salt, co-crystal, amorphous form, or crystalline form described herein (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

Relative amounts of the active ingredient, the pharmaceutically acceptable excipient, and/or any additional ingredients in a pharmaceutical composition of the present disclosure will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol. Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the salts, co-crystals, amorphous forms, and crystalline forms described herein are mixed with solubilizing agents such as Cremophor™, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the salts, co-crystals, amorphous forms, and crystalline forms described herein with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a salt, co-crystal, amorphous form, or crystalline form described herein may include ointments, pastes, creams, lotions, gels, powders, solutions, sprays, inhalants and/or patches. Generally, the active ingredient is admixed under sterile conditions with a pharmaceutically acceptable carrier and/or any desired preservatives and/or buffers as can be required. Additionally, the present disclosure encompasses the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A provided pharmaceutical composition can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A provided pharmaceutical composition can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A provided pharmaceutical composition can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The salts, co-crystals, amorphous forms, and crystalline forms provided herein are typically formulated in dosage unit form for ease of administration and uniformity of dosage. It will be understood, however, that the total daily usage of provided compositions will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The salts, co-crystals, amorphous forms, crystalline forms, and pharmaceutical compositions described herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a salt, co-crystal, amorphous form, or crystalline form required to achieve an effective amount will vary from subject to subject, depending, for example, on species, age, and general condition of a subject, severity of the side effects or disorder, identity of the particular salt, co-crystal, amorphous form, or crystalline form, mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a salt, co-crystal, amorphous form, or crystalline form described herein for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a salt, co-crystal, amorphous form, or crystalline form per unit dosage form.

In certain embodiments, a salt, co-crystal, amorphous form, or crystalline form described herein may be administered at dosage levels sufficient to deliver from about 0.001 mg/kg to about 1000 mg/kg, from about 0.01 mg/kg to about mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect.

In some embodiments, a salt, co-crystal, amorphous form, or crystalline form described herein is administered one or more times per day, for multiple days. In some embodiments, the dosing regimen is continued for days, weeks, months, or years.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A salt, co-crystal, amorphous form, crystalline form, or pharmaceutical composition described herein can be administered in combination with one or more additional therapeutically active agents. In certain embodiments, a salt, co-crystal, amorphous form, crystalline form, or pharmaceutical composition described herein is administered in combination with one or more additional therapeutically active agents that improve its bioavailability, reduce and/or modify its metabolism, inhibit its excretion, and/or modify its distribution within the body. It will also be appreciated that the therapy employed may achieve a desired effect for the same disorder, and/or it may achieve different effects.

A salt, co-crystal, amorphous form, crystalline form, or pharmaceutical composition described herein can be administered concurrently with, prior to, or subsequent to, one or more additional therapeutically active agents. In certain embodiments, the additional therapeutically active agent is a salt, co-crystal, amorphous form, or crystalline form described herein. In certain embodiments, the additional therapeutically active agent is not a salt, co-crystal, amorphous form, or crystalline form described herein. In general, each agent will be administered at a dose and/or on a time schedule determined for that agent. The additional therapeutically active agent utilized in this combination can be administered together in a single composition or administered separately in different compositions. The particular combination to employ in a regimen will take into account compatibility of a salt, co-crystal, amorphous form, or crystalline form described herein with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the present disclosure are kits (e.g., pharmaceutical packs). The kits provided may comprise a salt, co-crystal, amorphous form, crystalline form, or pharmaceutical composition described herein, and a container (e.g., a vial, ampule, bottle, syringe, and/or dispenser package, or other suitable container). In some embodiments, provided kits may optionally further include a second container comprising a pharmaceutical excipient for dilution or suspension of a salt, co-crystal, amorphous form, crystalline form, or pharmaceutical composition described herein. In some embodiments, a salt, co-crystal, amorphous form, crystalline form, or pharmaceutical composition described herein provided in the container and the second container are combined to form one unit dosage form. In some embodiments, a provided kits further includes instructions for use.

The salts, co-crystals, amorphous forms, crystalline forms, and pharmaceutical compositions described herein are generally useful for the inhibition of CARM1. In some embodiments, the CARM1 is human CARM1. In some embodiments, methods of treating CARM1-mediated disorder in a subject are provided which comprise administering an effective amount of a salt, co-crystal, amorphous form, crystalline form, or pharmaceutical composition described herein, to a subject in need of treatment. In certain embodiments, the effective amount is a therapeutically effective amount. In certain embodiments, the effective amount is a prophylactically effective amount. In certain embodiments, the subject is suffering from a CARM1-mediated disorder. In certain embodiments, the subject is susceptible to a CARM1-mediated disorder.

As used herein, the term “CARM1-mediated disorder” means any disease, disorder, or other pathological condition in which CARM1 is known to play a role. Accordingly, in some embodiments, the present disclosure relates to treating or lessening the severity of one or more diseases in which CARM1 is known to play a role.

In some embodiments, the present disclosure provides a method of inhibiting CARM1 comprising contacting CARM1 with an effective amount of a salt, co-crystal, amorphous form, crystalline form, or pharmaceutical composition described herein. The CARM1 may be purified or crude, and may be present in a cell, tissue, or subject. Thus, such methods encompass both inhibition of in vitro and in vivo CARM1 activity. In certain embodiments, the method is an in vitro method, e.g., such as an assay method. It will be understood by one of ordinary skill in the art that inhibition of CARM1 does not necessarily require that all of the CARM1 be occupied by an inhibitor at once. Exemplary levels of inhibition of CARM1 include at least 10% inhibition, about 10% to about 25% inhibition, about 25% to about 50% inhibition, about 50% to about 75% inhibition, at least 50% inhibition, at least 75% inhibition, at least 80% inhibition, at least 90% inhibition, and greater than 90% inhibition.

In some embodiments, provided is a method of inhibiting CARM1 activity in a subject in need thereof comprising administering to the subject an effective amount of a salt, co-crystal, amorphous form, crystalline form, or pharmaceutical composition described herein.

In certain embodiments, provided is a method of modulating gene expression or activity in a cell which comprises contacting a cell with an effective amount of a salt, co-crystal, amorphous form, crystalline form, or pharmaceutical composition described herein. In certain embodiments, the cell in culture in vitro. In certain embodiments, the cell is in an animal, e.g., a human. In certain embodiments, the cell is in a subject in need of treatment.

In certain embodiments, provided is a method of modulating transcription in a cell which comprises contacting a cell with an effective amount of a salt, co-crystal, amorphous form, crystalline form, or pharmaceutical composition described herein. In certain embodiments, the cell in culture in vitro. In certain embodiments, the cell is in an animal, e.g., a human. In certain embodiments, the cell is in a subject in need of treatment.

In certain embodiments, a method is provided of selecting a therapy for a subject having a disease associated with CARM1-mediated disorder or mutation comprising the steps of determining the presence of CARM1-mediated disorder or gene mutation in the CARM1 gene or and selecting, based on the presence of CARM1-mediated disorder a gene mutation in the CARM1 gene a therapy that includes the administration of a salt, co-crystal, amorphous form, or crystalline form described herein. In certain embodiments, the disease is a proliferative disorder. In certain embodiments, the disease is cancer.

In certain embodiments, a method of treatment is provided for a subject in need thereof comprising the steps of determining the presence of CARM1-mediated disorder or a gene mutation in the CARM1 gene and treating the subject in need thereof, based on the presence of a CARM1-mediated disorder or gene mutation in the CARM1 gene with a therapy that includes the administration of a salt, co-crystal, amorphous form, or crystalline form described herein. In certain embodiments, the subject is a cancer patient.

In some embodiments, a salt, co-crystal, amorphous form, crystalline form, or pharmaceutical composition described herein is useful in treating a proliferative disorder, such as cancer. For example, while not being bound to any particular mechanism, protein arginine methylation by CARM1 is a modification that has been implicated in signal transduction, gene transcription, DNA repair and mRNA splicing, among others; and overexpression of CARM1 within these pathways is often associated with various cancers. Thus, the salts, co-crystals, amorphous forms, crystalline forms, and pharmaceutical compositions, which inhibit the action of PRMTs, and specifically CARM1, are effective in the treatment of cancer.

In some embodiments, the salts, co-crystals, amorphous forms, crystalline forms, and pharmaceutical compositions described herein are effective in treating cancer through the inhibition of CARM1. For example, CARM1 levels have been shown to be elevated in castration-resistant prostate cancer (CRPC) (e.g., see Di Lorenzo et al., Drugs (2010) 70:983-1000), as well as in aggressive breast tumors (Hong et al., Cancer 2004 101, 83-89; El Messaoudi et al., Proc. Natl. Acad. Sci. U.S.A. 2006, 103, 13351-13356; Majumder et al., Prostate 2006 66, 1292-1301). Thus, in some embodiments, inhibitors of CARM1, such as such as the salts, co-crystals, amorphous forms, and crystalline forms described herein, are useful in treating cancers associated with aberrant CARM1 activity, e.g., CARM1 overexpression or aberrant protein methylation. For example, aberrant CARM1 activity has been found in prostate cancer (e.g., see Hong et al., Cancer (2004), 101:83-89); plays a coactivator role in the dysragulation of beta-catenin activity in colorectal cancer (e.g., see Ou et al., Mol. Cancer Res. (2011) 9:660); and has been linked to estrogen signaling and estrogen related cancers such as breast cancer (see, e.g., Teyssiewr et al., Trends in Endocrinology and Metabolism (2010) 21:181-189). CARM1 has also been shown to affect estrogen receptor alpha (ER-alpha) dependent breast cancer cell differentiation and proliferation (Al-Dhaheri et al., Cancer Res. 2011 71, 2118-2128), thus in some aspects CARM1 inhibitors, such as the salts, co-crystals, amorphous forms, and crystalline forms described herein, are useful in treating ERα-dependent breast cancer by inhibiting cell differentiation and proliferation. In another example, CARM1 has been shown to be recruited to the promoter of E2F1 (which encodes a cell cycle regulator) as a transcriptional co-activator (Frietze et al., Cancer Res. 2008 68, 301-306). Thus, CARM1-mediated upregulation of E2F1 expression may contribute to cancer progression and chemoresistance as increased abundance of E2F1 triggers invasion and metastasis by activating growth receptor signaling pathways, which in turn promote an antiapoptotic tumor environment (Engelmann and Püitzer, Cancer Res 2012 72; 571). Accordingly, in some embodiments, the inhibition of CARM1, e.g., by the salts, co-crystals, amorphous forms, crystalline forms, and pharmaceutical compositions described herein, is useful in treating cancers associated with E2F1 upregulation, e.g., such as lung cancer (see, e.g., Eymin et al., Oncogene (2001) 20:1678-1687), and breast cancer (see, e.g., Brietz et al., Cancer Res. (2008) 68:301-306). Thus, without being bound by any particular mechanism, the inhibition of CARM1, e.g., by the salts, co-crystals, amorphous forms, crystalline forms, and pharmaceutical compositions described herein, is beneficial in the treatment of cancer. CARM1 overexpression has also been demonstrated to be elevated in 75% of colorectal cancers (Kim et al., BMC Cancer, 10, 197). It has been additionally been determined that depletion of CARM1 in WNT/β-catenin dysregulated colorectal cancer suppressed anchorage independent growth (Ou et al., Mol. Cancer. Res., 2011 9, 660-670). This, in some embodiments, the inhibition of CARM1, e.g. by the salts, co-crystals, amorphous forms, crystalline forms, and pharmaceutical compositions described herein, is useful in colorectal cancer associated with elevated CARM1 expression or dysregulated WNT/β-catenin signaling.

In some embodiments, the salts, co-crystals, amorphous forms, crystalline forms, and pharmaceutical compositions described herein are useful for treating a cancer including, but not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenström's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic andenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer, and vulvar cancer (e.g., Paget's disease of the vulva).

In certain embodiments, the cancer is a solid cancer. In certain embodiments, the cancer is a liquid cancer. In certain embodiments, the cancer is breast cancer, prostate cancer, colorectal cancer, or a hematopoietic cancer (e.g., multiple myeloma).

CARM1 is also the most abundant PRMT expressed in skeletal muscle cells, and has been found to selectively control the pathways modulating glycogen metabolism, and associated AMPK (AMP-activated protein kinase) and p38 MAPK (mitogen-activated protein kinase) expression. See, e.g., Wang et al., Biochem (2012) 444:323-331. Thus, in some embodiments, inhibitors of CARM1, such as the salts, co-crystals, amorphous forms, and crystalline forms described herein, are useful in treating metabolic disorders, e.g., for example skeletal muscle metabolic disorders, e.g., glycogen and glucose metabolic disorders. Exemplary skeletal muscle metabolic disorders include, but are not limited to, Acid Maltase Deficiency (Glycogenosis type 2; Pompe disease), Debrancher deficiency (Glycogenosis type 3), Phosphorylase deficiency (McArdle's; GSD 5), X-linked syndrome (GSD9D), Autosomal recessive syndrome (GSD9B), Tarui's disease (Glycogen storage disease VII; GSD 7), Phosphoglycerate Mutase deficiency (Glycogen storage disease X; GSDX; GSD 10), Lactate dehydrogenase A deficiency (GSD 11), Branching enzyme deficiency (GSD 4), Aldolase A (muscle) deficiency, β-Enolase deficiency, Triosephosphate isomerase (TIM) deficiency, Lafora's disease (Progressive myoclonic epilepsy 2), Glycogen storage disease (Muscle, Type 0, Phosphoglucomutase 1 Deficiency (GSD 14)), and Glycogenin Deficiency (GSD 15).

In another aspect, the present disclosure provides uses of a salt, co-crystal, amorphous form, crystalline form, or pharmaceutical composition described herein in a method described herein.

In another aspect, the present disclosure provides salts, co-crystals, amorphous forms, crystalline forms, and pharmaceutical compositions described herein for use in a method described herein.

### EXAMPLES

In order that the invention described herein may be more fully understood, the following examples are set forth. These examples are for illustrative purposes only and are not to be construed as limiting this invention in any manner.

### Example 1

### Preparation of the Solid Forms

All reagents (e.g., the acids and solvents described herein) were purchased from commercial sources (e.g., Sigma Aldrich) at the Analytical Reagent grade and were used without purification.

Preparation of Form A

In an exemplary experiment, Form A was prepared as a white solid according to the method of preparing compound 109-3 as described in International PCT Application, PCT/US2014/028463, filed Mar. 14, 2014.

Preparation of Form G-A

In an exemplary experiment, to a glass vial was added 4.96 mg of gentisic acid, followed by 1.0 mL of a solution of Forma A in MeOH (at a concentration of 20 mg/mL). A clear solution resulted. The clear solution was agitated at 5° C. for 50 hours. No precipitate formed. The resulting solution was allowed to slowly evaporate at room temperature. A precipitate formed. The precipitate was filtered and dried at 50° C. in vacuo for 2 hours to give Form G-A (e.g., Sample 807304-06-A22) as slightly grey crystals.

In another exemplary experiment, to a glass vial was added 4.96 mg of gentisic acid, followed by 1.0 mL of a solution of Forma A in acetonitrile (at a concentration of 20 mg/mL). A clear solution resulted. The clear solution was agitated at 5° C. for 50 hours. No precipitate formed. The resulting solution was allowed to slowly evaporate at room temperature. A precipitate formed. The precipitate was filtered and dried at 50° C. in vacuo for 2 hours to give Form G-A (e.g., Samples 807304-06-D22, 807304-06-C22, and 807304-06-B22) as slightly grey crystals.

In another exemplary experiment, to a glass vial was added 4.96 mg of gentisic acid, followed by 1.0 mL of a solution of Forma A in acetone (at a concentration of 20 mg/mL). A clear solution resulted. The clear solution was agitated at 5° C. for 50 hours. No precipitate formed. The resulting solution was allowed to slowly evaporate at room temperature. A precipitate formed. The precipitate was filtered and dried at 50° C. in vacuo for 2 hours to give Form G-A (e.g., Samples 807304-06-D22, 807304-06-C22, and 807304-06-B22) as slightly grey crystals.

In another exemplary experiment, to a glass vial was added 4.96 mg of gentisic acid, followed by 1.0 mL of a solution of Forma A in THF (at a concentration of 20 mg/mL). A clear solution resulted. The clear solution was agitated at 5° C. for 50 hours. No precipitate formed. The resulting solution was allowed to slowly evaporate at room temperature. A precipitate formed. The precipitate was filtered and dried at 50° C. in vacuo for 2 hours to give Form G-A (e.g., Samples 807304-06-D22, 807304-06-C22, and 807304-06-B22) as slightly grey crystals.

Preparation of Form G-B

In an exemplary experiment, under a nitrogen atmosphere, Form G-A (e.g., sample 807304-06-B22) was heated to 100° C. using a thermogravimetric analyzer and then cooled to room temperature to give Form G-B (e.g., Samples 807304-18-A and 807304-19-A) as slightly grey crystals.

Preparation of Form G-C

In an exemplary experiment, to a glass vial was added 4.96 mg of gentisic acid, followed by 1.0 mL of a solution of Forma A in a mixture of n-heptane and acetone (1:1 by volume; at a concentration of 20 mg/mL). A clear solution resulted. The clear solution was agitated at 5° C. A precipitate formed. The precipitate was filtered and dried at 50° C. in vacuo to give Form G-C (e.g., Sample 807304-20-A1) as a white powder.

In another exemplary experiment, to a glass vial was added 4.96 mg of gentisic acid, followed by 1.0 mL of a solution of Forma A in a mixture of n-heptane and acetone (1.5:1 by volume; at a concentration of 20 mg/mL). A clear solution resulted. A trace amount of Form G-A was added. The resulting mixture was agitated at 5° C. A precipitate formed. The precipitate was filtered and dried at 50° C. in vacuo to give Form G-C (e.g., Samples 807304-21-Al and 807304-22-A3) as a white powder.

In another exemplary experiment, to a glass vial was added 4.96 mg of gentisic acid, followed by 1.0 mL of a solution of Forma A in a mixture of n-heptane and acetone (1.5:1 by volume; at a concentration of 20 mg/mL). A clear solution resulted. The clear solution was agitated at 5° C. A precipitate formed. The precipitate was filtered and dried at 50° C. in vacuo to give Form G-C (e.g., Sample 807304-22-A4) as a white powder.

In another exemplary experiment, to a glass vial was added 4.96 mg of gentisic acid, followed by 1.0 mL of a solution of Forma A in a mixture of n-heptane and THF (1:1 by volume; at a concentration of 20 mg/mL). A clear solution resulted. The clear solution was agitated at 5° C. A precipitate formed. The precipitate was filtered and dried at 50° C. in vacuo to give Form G-C (e.g., Sample 807304-20-A2) as a white powder.

### Example 2

### Characterization of the Solid Forms

X-ray Powder Diffraction (XRPD)

XRPD was performed with Panalytical Empyrean XRPD on a Si single crystal holder. The 2θ (2 theta) position was calibrated against Panalytical 640 Si powder standard. Exemplary XRPD parameters used in the experiments are as shown in Table 5 below.

TABLE 5

| Parameters | Settings/Values (Reflection Mode) |
|---|---|
| X-Ray wavelength | Cu, kα, |
| | Kα1 (Å): 1.540598, Kα2 (Å): 1.544426 |
| | Kα2/Kα1 intensity ratio: 0.50 |
| X-Ray tube setting | 45 kV, 40 mA |
| Divergence slit | Automatic |
| Scan mode | Continuous |
| Scan range (2 theta, °) | 2-40 |
| Step size (2 theta, °) | 0.0170 |
| Scan speed (°/min) | 10. |

Thermal Gravimetric Analysis (TGA) and Differential Scanning Calorimetry (DSC)

TGA was conducted at 10° C./min ramping from RT to desired temperature in open platinum pans using a TA Instruments Q5000 TGA. The temperature was calibrated using nickel and the weight using TA-supplied standard weights and verified against calcium oxalate monohydrate dehydration and decomposition.

DSC was performed with a TA instruments Q2000 DSC in crimped Al pan. The temperature and heat flow were calibrated against indium melting.

Exemplary parameters for TGA and DSC used in the experiments are as shown in Table 6 below.

TABLE 6

| | TGA | DSC |
|---|---|---|
| Temperature range | RT-300° C. | 25° C.-250° C. |
| Ramp rate | 10° C./min | 10° C./min |
| Purge gas | N₂ | N₂ |
| Pan type | Platinum, open | Aluminum, crimped. |

Proton Nuclear Magnetic Resonance (¹H-NMR)

Solution ¹H-NMR was collected on Bruker 400 MHz NMR Spectrometer using DMSO-d₆ or CDCl₃ as the solvent. Chemical shifts are reported in ppm with the residual solvent resonance as the internal standard (e.g., CHCl₃: δ 7.26; DMSO: δ 2.50).

Thermodynamic Solubility

In an exemplary experiment, the thermodynamic solubilities of the solid forms described herein were determined at room temperature (RT, 25±3° C.).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. An amorphous form A (Form A) of compound 109-3 of the formula:

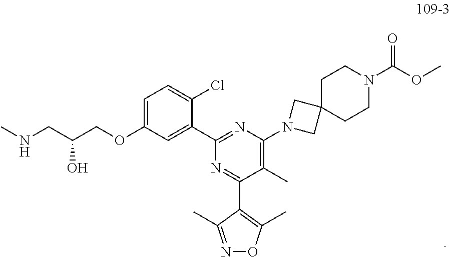

2. The amorphous form A of claim 1, wherein the amorphous form A is substantially free of impurities.

3. The amorphous form A of any one of claims 1-2, wherein the amorphous form A is substantially free of crystalline forms of compound 109-3.

4. The amorphous form A of any one of claims 1-3, wherein the amorphous form A is characterized by an X-ray powder diffraction (XRPD) pattern substantially similar to the one depicted in FIG. 1A.

5. The amorphous form A of any one of claims 1-4, wherein the amorphous form A is characterized by a differential scanning calorimetry (DSC) thermogram substantially similar to the one depicted in FIG. 1B.

6. The amorphous form A of any one of claims 1-5, wherein the amorphous form A is characterized by a differential scanning calorimetry (DSC) thermogram comprising an endotherm comprising a glass-transition temperature (T_{g}) of 65.1±2° C.

7. The amorphous form A of any one of claims 1-6, wherein the amorphous form A is characterized by a differential scanning calorimetry (DSC) thermogram comprising an endotherm comprising a midpoint temperature of 68.1±2° C.

8. The amorphous form A of any one of claims 1-7, wherein the amorphous form A is characterized by a differential scanning calorimetry (DSC) thermogram comprising an endotherm comprising a peak temperature (Tₘₐₓ) of 70.8±2° C.

9. The amorphous form A of any one of claims 1-8, wherein the amorphous form A is characterized by a differential scanning calorimetry (DSC) thermogram comprising a specific heat (Cₚ) of about 0.34 J/(g·° C.).

10. The amorphous form A of any one of claims 1-9, wherein the amorphous form A is characterized by a thermogravimetric analysis (TGA) thermogram substantially similar to the one depicted in FIG. 1B.

11. The amorphous form A of any one of claims 1-10, wherein the amorphous form A is characterized by a thermogravimetric analysis (TGA) thermogram comprising a weight loss of about 2.9% up to 150° C.

12. The amorphous form A of any one of claims 1-11, wherein the amorphous form A has one or more thermodynamic solubilities as shown in Table 1.

13. A crystalline form G-C (Form G-C) of compound 109-3 of the formula: 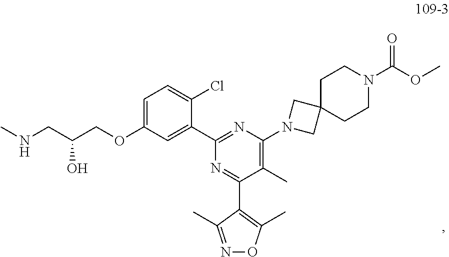
wherein the crystalline form G-C comprises gentisic acid.

14. The crystalline form G-C of claim 13, wherein the crystalline form G-C is obtained by recrystallization of compound 109-3 from a mixture of n-heptane and acetone.

15. The crystalline form G-C of claim 13, wherein the crystalline form G-C is obtained by recrystallization of compound 109-3 from a mixture of n-heptane and tetrahydrofuran (THF).

16. The crystalline form G-C of any one of claims 13-15, wherein the crystalline form G-C is a co-crystal of compound 109-3 and gentisic acid.

17. The crystalline form G-C of any one of claims 13-15, wherein the crystalline form G-C is a gentisate of compound 109-3.

18. The crystalline form G-C of any one of claims 13-17, wherein the molar ratio of gentisic acid to compound 109-3 in the crystalline form G-C is about 1:1.

19. The crystalline form G-C of any one of claims 13-18, wherein the crystalline form G-C is a solvate.

20. The crystalline form G-C of claim 19, wherein the crystalline form G-C is a hydrate.

21. The crystalline form G-C of any one of claims 13-20, wherein the crystalline form G-C is substantially free of impurities.

22. The crystalline form G-C of any one of claims 13-21, wherein the crystalline form G-C is characterized by an X-ray powder diffraction (XRPD) pattern substantially similar to the one depicted in FIG. 4A.

23. The crystalline form G-C of any one of claims 13-22, wherein the crystalline form G-C is characterized by an XRPD pattern comprising three or more characteristic peaks, expressed in degrees 2-theta (±0.2), independently selected from the group consisting of 17.86, 19.44, 19.24, 18.65, 20.49, 23.88, 11.43, 15.83, and 23.45.

24. The crystalline form G-C of any one of claims 13-22, wherein the crystalline form G-C is characterized by an XRPD pattern comprising four or more characteristic peaks, expressed in degrees 2-theta (±0.2), independently selected from the group consisting of 17.86, 19.44, 19.24, 18.65, 20.49, 23.88, 11.43, 15.83, and 23.45.

25. The crystalline form G-C of any one of claims 13-22, wherein the crystalline form G-C is characterized by an XRPD pattern comprising five or more characteristic peaks, expressed in degrees 2-theta (±0.2), independently selected from the group consisting of 17.86, 19.44, 19.24, 18.65, 20.49, 23.88, 11.43, 15.83, and 23.45.

26. The crystalline form G-C of any one of claims 13-22, wherein the crystalline form G-C is characterized by an XRPD pattern comprising six or more characteristic peaks, expressed in degrees 2-theta (±0.2), independently selected from the group consisting of 17.86, 19.44, 19.24, 18.65, 20.49, 23.88, 11.43, 15.83, and 23.45.

27. The crystalline form G-C of any one of claims 13-22, wherein the crystalline form G-C is characterized by an XRPD pattern comprising seven or more characteristic peaks, expressed in degrees 2-theta (±0.2), independently selected from the group consisting of 17.86, 19.44, 19.24, 18.65, 20.49, 23.88, 11.43, 15.83, and 23.45.

28. The crystalline form G-C of any one of claims 13-22, wherein the crystalline form G-C is characterized by an XRPD pattern comprising eight or more characteristic peaks, expressed in degrees 2-theta (±0.2), independently selected from the group consisting of 17.86, 19.44, 19.24, 18.65, 20.49, 23.88, 11.43, 15.83, and 23.45.

29. The crystalline form G-C of any one of claims 13-28, wherein the crystalline form G-C is characterized by a differential scanning calorimetry (DSC) thermogram substantially similar to the one depicted in FIG. 4B.

30. The crystalline form G-C of any one of claims 13-29, wherein the crystalline form G-C is characterized by a differential scanning calorimetry (DSC) thermogram comprising an endotherm comprising an onset temperature (Tₘ) of 154.4±2° C.

31. The crystalline form G-C of any one of claims 13-30, wherein the crystalline form G-C is characterized by a differential scanning calorimetry (DSC) thermogram comprising an endotherm comprising a peak temperature (Tₘₐₓ) of 167.9±2° C.

32. The crystalline form G-C of claim 31, wherein the crystalline form G-C is characterized by a differential scanning calorimetry (DSC) thermogram further comprising another endotherm comprising a peak temperature (Tₘₐₓ) of 56.9±2° C.

33. The crystalline form G-C of any one of claims 13-32, wherein the crystalline form G-C is characterized by a thermogravimetric analysis (TGA) thermogram substantially similar to the one depicted in FIG. 4B.

34. The crystalline form G-C of any one of claims 13-33, wherein the crystalline form G-C is characterized by a thermogravimetric analysis (TGA) thermogram comprising a weight loss of about 2.6% up to 150° C.

35. A crystalline form G-A (Form G-A) of compound 109-3 of the formula: 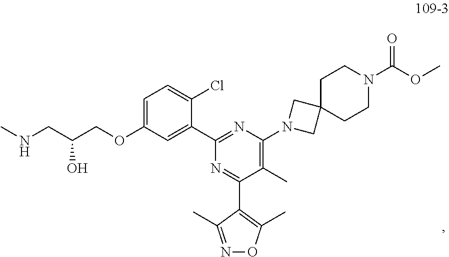
wherein the crystalline form G-A comprises gentisic acid.

36. The crystalline form G-A of claim 35, wherein the crystalline form G-A is obtained by recrystallization of compound 109-3 from methanol.

37. The crystalline form G-A of claim 35, wherein the crystalline form G-A is obtained by recrystallization of compound 109-3 from acetonitrile.

38. The crystalline form G-A of claim 35, wherein the crystalline form G-A is obtained by recrystallization of compound 109-3 from acetone.

39. The crystalline form G-A of claim 35, wherein the crystalline form G-A is obtained by recrystallization of compound 109-3 from tetrahydrofuran (THF).

40. The crystalline form G-A of any one of claims 35-39, wherein the crystalline form G-A is a co-crystal of compound 109-3 and gentisic acid.

41. The crystalline form G-A of any one of claims 35-39, wherein the crystalline form G-A is a gentisate of compound 109-3.

42. The crystalline form G-A of any one of claims 35-41, wherein the molar ratio of gentisic acid to compound 109-3 in the crystalline form G-A is about 1:1.

43. The crystalline form G-A of any one of claims 35-42, wherein the crystalline form G-A is a solvate.

44. The crystalline form G-A of claim 43, wherein the crystalline form G-A is a hydrate.

45. The crystalline form G-A of any one of claims 35-44, wherein the crystalline form G-A is substantially free of impurities.

46. The crystalline form G-A of any one of claims 35-45, wherein the crystalline form G-A is characterized by an X-ray powder diffraction (XRPD) pattern substantially similar to the one depicted in FIG. 2A.

47. The crystalline form G-A of any one of claims 35-46, wherein the crystalline form G-A is characterized by an XRPD pattern comprising three or more characteristic peaks, expressed in degrees 2-theta (±0.2), independently selected from the group consisting of 20.51, 13.37, 9.13, 19.64, 13.04, 19.86, 18.30, 9.49, and 18.68.

48. The crystalline form G-A of any one of claims 35-46, wherein the crystalline form G-A is characterized by an XRPD pattern comprising four or more characteristic peaks, expressed in degrees 2-theta (±0.2), independently selected from the group consisting of 20.51, 13.37, 9.13, 19.64, 13.04, 19.86, 18.30, 9.49, and 18.68.

49. The crystalline form G-A of any one of claims 35-46, wherein the crystalline form G-A is characterized by an XRPD pattern comprising five or more characteristic peaks, expressed in degrees 2-theta (±0.2), independently selected from the group consisting of 20.51, 13.37, 9.13, 19.64, 13.04, 19.86, 18.30, 9.49, and 18.68.

50. The crystalline form G-A of any one of claims 35-46, wherein the crystalline form G-A is characterized by an XRPD pattern comprising six or more characteristic peaks, expressed in degrees 2-theta (±0.2), independently selected from the group consisting of 20.51, 13.37, 9.13, 19.64, 13.04, 19.86, 18.30, 9.49, and 18.68.

51. The crystalline form G-A of any one of claims 35-46, wherein the crystalline form G-A is characterized by an XRPD pattern comprising seven or more characteristic peaks, expressed in degrees 2-theta (±0.2), independently selected from the group consisting of 20.51, 13.37, 9.13, 19.64, 13.04, 19.86, 18.30, 9.49, and 18.68.

52. The crystalline form G-A of any one of claims 35-46, wherein the crystalline form G-A is characterized by an XRPD pattern comprising eight or more characteristic peaks, expressed in degrees 2-theta (±0.2), independently selected from the group consisting of 20.51, 13.37, 9.13, 19.64, 13.04, 19.86, 18.30, 9.49, and 18.68.

53. The crystalline form G-A of any one of claims 35-52, wherein the crystalline form G-A is characterized by a differential scanning calorimetry (DSC) thermogram substantially similar to the one depicted in FIG. 2B.

54. The crystalline form G-A of any one of claims 35-53, wherein the crystalline form G-A is characterized by a differential scanning calorimetry (DSC) thermogram comprising an endotherm comprising an onset temperature (Tₘ) of 178.3±2° C.

55. The crystalline form G-A of any one of claims 35-54, wherein the crystalline form G-A is characterized by a differential scanning calorimetry (DSC) thermogram comprising an endotherm comprising a peak temperature (Tₘₐₓ) of 186.2±2° C.

56. The crystalline form G-A of claim 55, wherein the crystalline form G-A is characterized by a differential scanning calorimetry (DSC) thermogram further comprising another endotherm comprising a peak temperature (Tₘₐₓ) of 96.0±2° C.

57. The crystalline form G-A of claim 55 or 56, wherein the crystalline form G-A is characterized by a differential scanning calorimetry (DSC) thermogram further comprising another endotherm comprising a peak temperature (Tₘₐₓ) of 81.4±2° C.

58. The crystalline form G-A of any one of claims 35-57, wherein the crystalline form G-A is characterized by a differential scanning calorimetry (DSC) thermogram comprising an enthalpy of the endothermic transition (ΔH) of about 49.78 J/g.

59. The crystalline form G-A of any one of claims 35-58, wherein the crystalline form G-A is characterized by a thermogravimetric analysis (TGA) thermogram substantially similar to the one depicted in FIG. 2B.

60. The crystalline form G-A of any one of claims 35-59, wherein the crystalline form G-A is characterized by a thermogravimetric analysis (TGA) thermogram comprising a weight loss of about 7.9% up to 100° C.

61. A crystalline form G-B (Form G-B) of compound 109-3 of the formula: 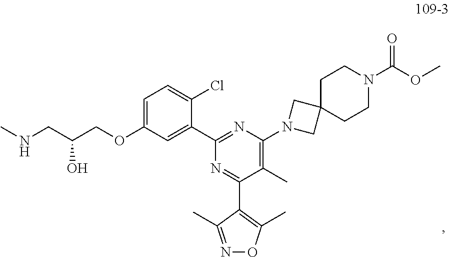
wherein the crystalline form G-B comprises gentisic acid.

62. The crystalline form G-B of claim 61, wherein the crystalline form G-B is obtained by heating a crystalline form of any one of claims 35-59 to about 100° C. and cooling to about 25° C.

63. The crystalline form G-B of any one of claims 61-62, wherein the crystalline form G-B is a co-crystal of compound 109-3 and gentisic acid.

64. The crystalline form G-B of any one of claims 61-62, wherein the crystalline form G-B is a gentisate of compound 109-3.

65. The crystalline form G-B of any one of claims 61-64, wherein the molar ratio of gentisic acid to compound 109-3 in the crystalline form G-B is about 1:1.

66. The crystalline form G-B of any one of claims 61-65, wherein the crystalline form G-B is a solvate.

67. The crystalline form G-B of claim 66, wherein the crystalline form G-B is a hydrate.

68. The crystalline form G-B of any one of claims 61-67, wherein the crystalline form G-B is substantially free of impurities.

69. The crystalline form G-B of any one of claims 61-68, wherein the crystalline form G-B is characterized by an X-ray powder diffraction (XRPD) pattern substantially similar to the one depicted in FIG. 3A.

70. The crystalline form G-B of any one of claims 61-69, wherein the crystalline form G-B is characterized by an XRPD pattern comprising three or more characteristic peaks, expressed in degrees 2-theta (±0.2), independently selected from the group consisting of 17.53, 15.87, 10.57, 20.92, 22.22, 18.67, 21.21, 20.34, and 27.06.

71. The crystalline form G-B of any one of claims 61-69, wherein the crystalline form G-B is characterized by an XRPD pattern comprising four or more characteristic peaks, expressed in degrees 2-theta (±0.2), independently selected from the group consisting of 17.53, 15.87, 10.57, 20.92, 22.22, 18.67, 21.21, 20.34, and 27.06.

72. The crystalline form G-B of any one of claims 61-69, wherein the crystalline form G-B is characterized by an XRPD pattern comprising five or more characteristic peaks, expressed in degrees 2-theta (±0.2), independently selected from the group consisting of 17.53, 15.87, 10.57, 20.92, 22.22, 18.67, 21.21, 20.34, and 27.06.

73. The crystalline form G-B of any one of claims 61-69, wherein the crystalline form G-B is characterized by an XRPD pattern comprising six or more characteristic peaks, expressed in degrees 2-theta (±0.2), independently selected from the group consisting of 17.53, 15.87, 10.57, 20.92, 22.22, 18.67, 21.21, 20.34, and 27.06.

74. The crystalline form G-B of any one of claims 61-69, wherein the crystalline form G-B is characterized by an XRPD pattern comprising seven or more characteristic peaks, expressed in degrees 2-theta (±0.2), independently selected from the group consisting of 17.53, 15.87, 10.57, 20.92, 22.22, 18.67, 21.21, 20.34, and 27.06.

75. The crystalline form G-B of any one of claims 61-69, wherein the crystalline form G-B is characterized by an XRPD pattern comprising eight or more characteristic peaks, expressed in degrees 2-theta (±0.2), independently selected from the group consisting of 17.53, 15.87, 10.57, 20.92, 22.22, 18.67, 21.21, 20.34, and 27.06.

76. The crystalline form G-B of any one of claims 61-75, wherein the crystalline form G-B is characterized by a differential scanning calorimetry (DSC) thermogram substantially similar to the one depicted in FIG. 3C.

77. The crystalline form G-B of any one of claims 61-76, wherein the crystalline form G-B is characterized by a differential scanning calorimetry (DSC) thermogram comprising an endotherm comprising an onset temperature (Tₘ) of 173.4±2° C.

78. The crystalline form G-B of any one of claims 61-77, wherein the crystalline form G-B is characterized by a differential scanning calorimetry (DSC) thermogram comprising an endotherm comprising a peak temperature (Tₘₐₓ) of 186.1±2° C.

79. The crystalline form G-B of claim 78, wherein the crystalline form G-B is characterized by a differential scanning calorimetry (DSC) thermogram further comprising another endotherm comprising a peak temperature (Tₘₐₓ) of 80.4±2° C.

80. The crystalline form G-B of claim 78 or 79, wherein the crystalline form G-B is characterized by a differential scanning calorimetry (DSC) thermogram further comprising another endotherm comprising a peak temperature (Tₘₐₓ) of 65.1±2° C.

81. The crystalline form G-B of any one of claims 61-80, wherein the crystalline form G-B is characterized by a thermogravimetric analysis (TGA) thermogram substantially similar to the one depicted in FIG. 3B.

82. The crystalline form G-B of any one of claims 61-81, wherein the crystalline form G-B is characterized by a thermogravimetric analysis (TGA) thermogram comprising a weight loss of about 3.2% up to 100° C.

83. The crystalline form G-B of any one of claims 61-80, wherein the crystalline form G-B is characterized by a thermogravimetric analysis (TGA) thermogram substantially similar to the one depicted in FIG. 3C.

84. The crystalline form G-B of any one of claims 61-81, wherein the crystalline form G-B is characterized by a thermogravimetric analysis (TGA) thermogram comprising a weight loss of about 2.7% up to 100° C.

85. A pharmaceutical composition comprising an amorphous form or crystalline form of any one of claims 1-84, and optionally a pharmaceutically acceptable excipient.

86. A kit comprising an amorphous form or crystalline form of any one of claims 1-84 or a pharmaceutical composition of claim 85, and instructions for using the amorphous form, crystalline form, or pharmaceutical composition.

87. A method of treating a CARM1 (co-activator-associated arginine methyltransferase 1)-mediated disorder, comprising administering to a subject in need thereof an effective amount of an amorphous form or crystalline form of any one of claims 1-84, or a pharmaceutical composition of claim 85.

88. The method of claim 87, wherein the CARM1-mediated disorder is a proliferative disorder.

89. The method of claim 88, wherein the CARM1-mediated disorder is cancer.

90. The method of claim 89, wherein the cancer is associated with E2F1 upregulation.

91. The method of claim 89 or 90, wherein the cancer is associated with aberrant CARM1 activity.

92. The method of any one of claims 89-91, wherein the cancer is breast cancer.

93. The method of claim 92, wherein the breast cancer is ERα-dependent breast cancer.

94. The method of any one of claims 89-91, wherein the cancer is prostate cancer.

95. The method of claim 94, wherein the prostate cancer is castration-resistant prostate cancer.

96. The method of any one of claims 89-91, wherein the cancer is colorectal cancer.

97. The method of claim 96, wherein the colorectal cancer is a colorectal cancer associated with dysregulated WNT/β-catenin signaling.

98. The method of claim 87, wherein the CARM1-mediated disorder is a metabolic disorder.
